(19) United States
(12) Patent Application Publication
(10) Pub. No.: US 2021/0317456 A1
(43) Pub. Date: Oct. 14, 2021

(54) Nucleic Acid Compounds for Binding Immunoglobulin G

(71) Applicant: SomaLogic, Inc., Boulder, CO (US)

(72) Inventors: Steven K. Wolk, Boulder, CO (US); Jessica Guillot, Boulder, CO (US); Erik Zimmerman, Bouler, CO (US); Michael Vrklijan, Boulder, CO (US); Allison Weiss, Boulder, CO (US); Daniel W. Drolet, Boulder, CO (US); Daniel J. Schneider, Boulder, CO (US); Sheri Wilcox, Boulder, CO (US); Nebojsa Janjic, Boulder, CO (US)

(73) Assignee: SomaLogic, Inc., Boulder, CO (US)

(21) Appl. No.: 17/284,556

(22) PCT Filed: Oct. 15, 2019
(86) PCT No.: PCT/US2019/056234

§ 371 Date: Apr. 12, 2021

Related U.S. Application Data
(60) Provisional application No. 62/750,958, filed on Oct. 26, 2018.
(60) Provisional application No. 62/745,503, filed on Oct. 15, 2018.

(51) Int. Cl. C12N 15/115; C07K 1/22

(52) U.S. Cl. CPC C12N 15/115; C07K 1/22; C12N 2310/315; C12N 2310/322; C12N 2310/335; C12N 2310/321

(57) ABSTRACT

Described herein are aptamers capable of binding to immunoglobulin G (IgG); compositions comprising an IgG binding aptamer with an IgG protein; and methods of making and using the same. Provided herein are also methods of releasing proteins bound to aptamers.

### CROSS-REFERENCE TO RELATED APPLICATIONS

This application claims the benefit of priority of U.S. Provisional Application No. 62/745,503, filed Oct. 15, 2018, and U.S. Provisional Application No. 62/750,958, filed Oct. 26, 2018, each of which is incorporated by reference herein in its entirety for any purpose.

### SEQUENCE LISTING

This application contains a Sequence Listing, which has been submitted electronically in ASCII format and is hereby incorporated by reference in its entirety. Said ASCII copy, created on Oct. 2, 2019, is named 2019-10-15_01137-0025-00PCT_ST25.txt and is 75 kilobytes in size.

### FIELD

The present disclosure relates generally to the field of nucleic acids, and more specifically, to aptamers capable of binding to immunoglobulin G (IgG) protein; compositions comprising an IgG binding aptamer; and methods of making and using the same.

### BACKGROUND

Human Immunoglobulin G (IgG) is used in numerous applications such as in monoclonal antibodies and Fc fusion proteins used for personalized therapies, detection strategies in Western blots, fluorescence microscopy and flow cytometry. Currently, IgG purification is performed using Protein A affinity, which is a commonly used affinity chromatography purification method.

The use of an aptamer reagent to capture an antibody or Fc fusion protein for affinity purification or to detect an antibody in other applications is advantageous. Aptamers provide an ideal alternative to protein A and antibodies and possess several key advantages, including lower molecular weight, which translates into a higher number of moles of target bound per gram; greater stability (both tolerance of temperature and pH conditions, and recoverability from non-ideal conditions); longer shelf-life without special requirements of cooling; lack of aggregation properties that can be a problem with antibodies; more cost effective and reproducible production; potential for greater specificity and affinity to target; and more easily modified and therefore “tunable” to a specific target or class of targets. The present disclosure meets such needs by providing aptamers having binding specificity to IgG-containing proteins.

### SUMMARY

In some embodiments, aptamers having binding specificity to IgG-containing proteins are provided.

In some embodiments, an aptamer comprises a nucleobase sequence selected from the group consisting of SEQ ID NOs: 1-6, 10-16, 18-34, 36-47, 48-57, 65-69, 71-74, 78, 79-84, 88-93, 96-98, 100-102 and 104-106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine. In some embodiments, an aptamer comprises the nucleobase sequence selected from SEQ ID NOs: 45, 46 and 47, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine. In some embodiments, an aptamer comprises the nucleobase sequence selected from SEQ ID NOs: 69, 74 and 78, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine. In some embodiments, an aptamer comprises the nucleobase sequence of SEQ ID NO: 106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

In some embodiments, an aptamer binds IgG with an affinity greater than 50 nM, or greater than 100 nM, or greater than 150 nM, or greater than 200 nM, or greater than 250 nM, or greater than 300 nM. In some embodiments, an aptamer binds IgG with an affinity less than 8 nM, or less than 7 nM, or less than 6 nM, or less than 5 nM, or less than 4 nM, or less than 3 nM, or less than 2 nM, or less than 1 nM.

In some embodiments, an aptamer comprises a C-5 modified pyrimidine containing nucleoside selected from the group consisting of 5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N—R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

In some embodiments, an aptamer comprises a C-5 modified pyrimidine containing nucleoside selected from a 5-(N-naphthylmethylcarboxyamide)-2′-deoxyuridine (NapdU) and a 5-(N-2-naphthylmethylcarboxyamide)-2′-deoxyuridine (2NapdU).

In some embodiments, the 5′-end of the nucleotide sequence of an aptamer further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides). In some embodiments, the 3′-end of the nucleotide sequence of an aptamer further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides). In some embodiments, an aptamer is provided wherein the 5′-end and the 3′-end, independently, of the nucleotide sequence further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

In some embodiments, an aptamer comprises a C-5 modified pyrimidine containing nucleoside which is a 5-(N-naphthylmethylcarboxyamide)-2′-deoxyuridine (NapdU). In some embodiments, an aptamer is provided comprising a C-5 modified pyrimidine containing nucleoside which is a 5-(N-2-naphthylmethylcarboxyamide)-2′-deoxyuridine (2NapdU).

In some embodiments, an aptamer binds an IgG protein selected from IgG1, IgG2, IgG3 and IgG4. In some embodiments, an aptamer binds an IgG protein selected from human IgG protein, monkey IgG protein, mouse IgG protein, cow IgG protein, goat IgG protein, sheep IgG protein and rabbit IgG protein.

In some embodiments, an aptamer is at least from 27 to 100 nucleotides in length (or from 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length).

In some embodiments, an aptamer is provided wherein at least one nucleotide of the nucleotide sequence comprises a 2′-O-methyl modification. In some embodiments, an aptamer is provided wherein at least one internucleoside linkage of the nucleotide sequence is a phosphorothioate.

In some embodiments, a composition is provided comprising an IgG protein and an aptamer comprising the nucleobase sequence selected from the group consisting of SEQ ID NOs: 1-6, 10-16, 18-34, 36-47, 48-57, 65-69, 71-74, 78, 79-84, 88-93, 96-98, 100-102 and 104-106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine. In some embodiments, a composition comprises an IgG protein and an aptamer comprising the nucleobase sequence selected from the group consisting of SEQ ID NOs: 45, 46 and 47, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine. In some embodiments, a composition comprises an IgG protein and an aptamer comprising the nucleobase sequence selected from the group consisting of SEQ ID NOs: 69, 74 and 78, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine. In some embodiments, a composition comprises an IgG protein and an aptamer comprising the nucleobase sequence selected from the group consisting of SEQ ID NO: 106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine. In some embodiments, a composition is provided wherein the nucleoside comprising the C-5 modified pyrimidine of the aptamer is selected from the group consisting of 5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N—R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

In embodiments, the C-5 modified pyrimidine is selected from a 5-(N-naphthylmethylcarboxyamide)-2′-deoxyuridine (NapdU) and a 5-(N-2-naphthylmethylcarboxyamide)-2′-deoxyuridine (2NapdU).

In some embodiments, a composition is provided comprising an IgG protein and an aptamer, wherein the 5′-end of the nucleotide sequence of the aptamer further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides). In some embodiments, a composition is provided comprising an IgG protein and an aptamer, wherein the 3′-end of the nucleotide sequence of the aptamer further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides). In some embodiments, a composition is provided comprising an IgG protein and an aptamer, wherein the 5′-end and the 3′-end, independently, of the nucleotide sequence of the aptamer further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

In some embodiments, a composition is provided comprising an IgG protein and an aptamer, wherein a nucleoside comprising a C-5 modified pyrimidine of the aptamer is a 5-(N-naphthylmethylcarboxyamide)-2′-deoxyuridine (NapdU). In some embodiments, a composition is provided comprising an IgG protein and an aptamer, wherein the C-5 modified pyrimidine containing nucleoside is a 5-(N-2-naphthylmethylcarboxyamide)-2′-deoxyuridine (2NapdU).

In some embodiments, a composition is provided comprising an IgG protein and an aptamer, wherein the aptamer is at least from 27 to 100 nucleotides in length (or from 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length).

In some embodiments, an aptamer is provided, wherein one or more P in the nucleobase sequence of the aptamer are a uracil. In some embodiments, each P in the nucleobase sequence of the aptamer is a C-5 modified pyrimidine comprising a napthyl substituent covalently linked via a linker to the C-5 position of the pyrimidine base. In some embodiments, the linker is selected from the group consisting of an amide linker, a carbonyl linker, a propynyl linker, an alkyne linker, an ester linker, a urea linker, a carbamate linker, a guanidine linker, an amidine linker, a sulfoxide linker, and a sulfone linker and a combination thereof.

In some embodiments, a composition is provided comprising an IgG protein and an aptamer, wherein one or more P positions of the aptamer are a uracil.

In some embodiments, a composition is provided comprising an IgG protein and an aptamer, wherein each P in the nucleobase sequence of the aptamer is a C-5 modified pyrimidine comprising a napthyl substituent covalently linked via a linker to the C-5 position of the pyrimidine base. In some embodiments, the linker is selected from the group consisting of an amide linker, a carbonyl linker, a propynyl linker, an alkyne linker, an ester linker, a urea linker, a carbamate linker, a guanidine linker, an amidine linker, a sulfoxide linker, and a sulfone linker and a combination thereof.

In some embodiments, a method is provided for purifying an IgG protein from a sample comprising the steps of: a) incubating the sample with an aptamer capable of binding IgG to produce an IgG protein-aptamer complex and b) eluting the IgG protein from the complex. In some embodiments, the elution is performed in the presence of benzamidine, an alkyl imidazolium derivative, or a combination thereof. In some embodiments, the elution is performed in the presence of benzamidine, an alkyl imidazolium derivative, or a combination thereof. In some embodiments, the alkyl imidazolium derivative has the resonance structure:

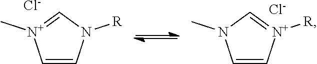

wherein R is selected from the group consisting of non-substituted alkyl, alkenyl, and benzyl. In some embodiments, R is selected from the group consisting of non-substituted C₁-C₁₂ alkyl, C₂-C₆ alkenyl, and benzyl. In some embodiments, R is selected from the group consisting of C₂-C₁₀ alkyl, C₂-C₄ alkenyl, and benzyl. In some embodiments, the alkyl imidazolium derivative is selected from the group consisting of 1-decyl-3-methylimidazolium chloride, 1-methyl-3-octylimidazolium chloride, 1-hexyl-3-methylimidazolium chloride, 1-benzyl-3-methylimidazolium chloride, 1-butyl-3-methylimidazolium chloride, and 1-allyl-3-methylimidazolium chloride.

In some embodiments, a method is provided for purifying a protein from a sample comprising the steps of: a) incubating the sample with an aptamer capable of binding the protein to produce a protein-aptamer complex and b) eluting the protein from the complex. In some embodiments, the elution is performed in the presence of benzamidine, an alkyl imidazolium derivative, or a combination thereof. In some embodiments, the alkyl imidazolium derivative has the resonance structure:

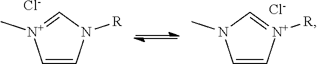

wherein R is selected from the group consisting of non-substituted alkyl, alkenyl, and benzyl. In some embodiments, R is selected from the group consisting of non-substituted C₁-C₁₂ alkyl, C₂-C₆ alkenyl, and benzyl. In some embodiments, R is selected from the group consisting of C₂-C₁₀ alkyl, C₂-C₄ alkenyl, and benzyl. In some embodiments, the alkyl imidazolium derivative is selected from the group consisting of 1-decyl-3-methylimidazolium chloride, 1-methyl-3-octylimidazolium chloride, 1-hexyl-3-methylimidazolium chloride, 1-benzyl-3-methylimidazolium chloride, 1-butyl-3-methylimidazolium chloride, and 1-allyl-3-methylimidazolium chloride.

In some embodiments, the protein retains activity following elution from the protein-aptamer complex.

In some embodiments, the aptamer comprises at least one C-5 modified pyrimidine. In some embodiments, a nucleoside comprising the C-5 modified pyrimidine is selected from the group consisting of
5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N—R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

In some embodiments, the aptamer comprises a detectable label. In some embodiments, the aptamer is bound to a solid support. In some embodiments, the aptamer comprises a member of a binding pair capable of being captured on a solid support. In some embodiments, the aptamer is biotinylated. In some embodiments, the solid support comprises streptavidin.

In some embodiments, the protein is an immunoglobulin protein. In some embodiments, the protein is a domain of an immunoglobulin protein. In some embodiments, the protein is an Fc region of an antibody or a Fab region of an antibody. In some embodiments, the protein is an IgA, an IgD, and IgE, and IgG or an IgM.

The foregoing and other objects, features, and advantages of the invention will become more apparent from the following detailed description, which proceeds with reference to the accompanying figures.

### BRIEF DESCRIPTION OF THE DRAWINGS

FIG. 1. Certain exemplary 5-position modified uricils and cytosines that may be incorporated into aptamers.

FIG. 2. Certain exemplary modifications that may be present at the 5-position of uracil or uridine and certain exemplary modified uridines. The chemical structure of the C-5 modification includes the exemplary amide linkage that links the modification to the 5-position of the uracil or uridine. The 5-position moieties shown include a benzyl moiety (e.g., Bn, PE and a PP), a naphthyl moiety (e.g., Nap, 2Nap, NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), a benzofuranyl moiety (e.g., BF), an indole moiety (e.g, Trp) and a hydroxypropyl moiety (e.g., Thr).

FIG. 3. Certain exemplary modifications that may be present at the 5-position of cytosine or cytidine and certain exemplary modified cytidines. The chemical structure of the C-5 modification includes the exemplary amide linkage that links the modification to the 5-position of the cytosine or cytidine. The 5-position moieties shown include a benzyl moiety (e.g., Bn, PE and a PP), a naphthyl moiety (e.g., Nap, 2Nap, NE, and 2NE) and a tyrosyl moiety (e.g., a Tyr).

### DETAILED DESCRIPTION

### Terms and Methods

Unless otherwise noted, technical terms are used according to conventional usage. Definitions of common terms in molecular biology may be found, for example, in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Aptamer: As used herein, “aptamer,” “nucleic acid ligand,” “SOMAmer,” “modified aptamer,” and “clone” are used interchangeably to refer to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule.

In some embodiments, the action is specific binding affinity for a target molecule, such target molecule being a three dimensional chemical structure other than a polynucleotide that binds to the aptamer through a mechanism which is independent of Watson/Crick base pairing or triple helix formation, wherein the aptamer is not a nucleic acid having the known physiological function of being bound by the target molecule. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target generally with a much higher degree of affinity than it binds to other, non-target, components in a mixture or sample. An “aptamer,” “SOMAmer,” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In some embodiments, the aptamers are prepared using a SELEX process as described herein, or known in the art.

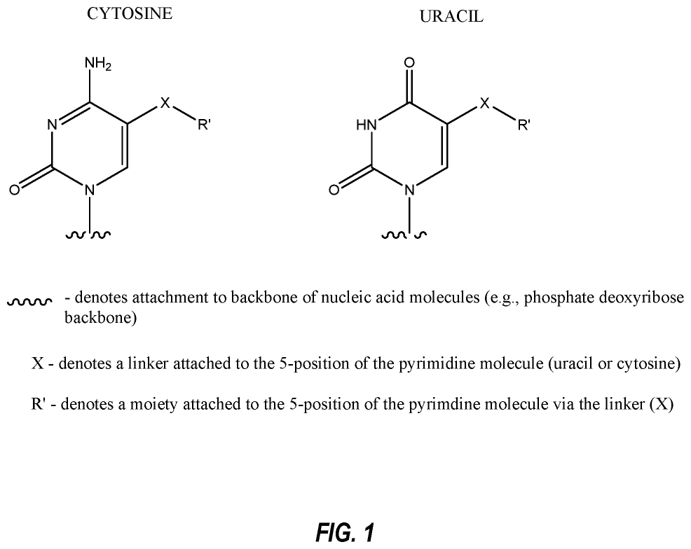

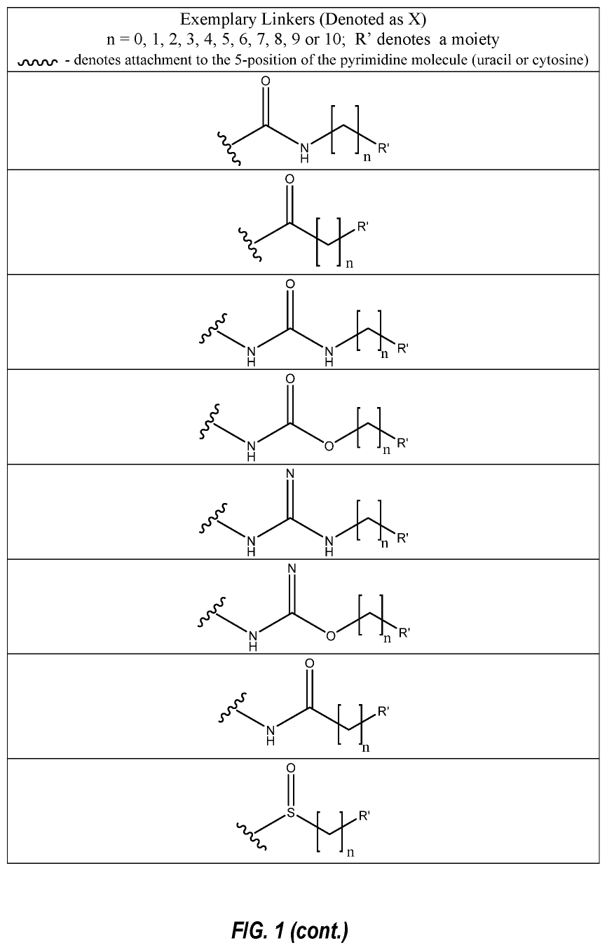

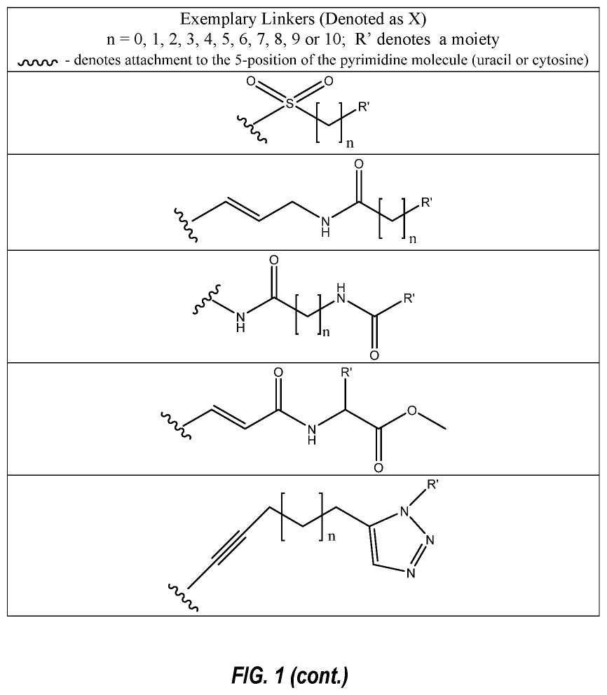

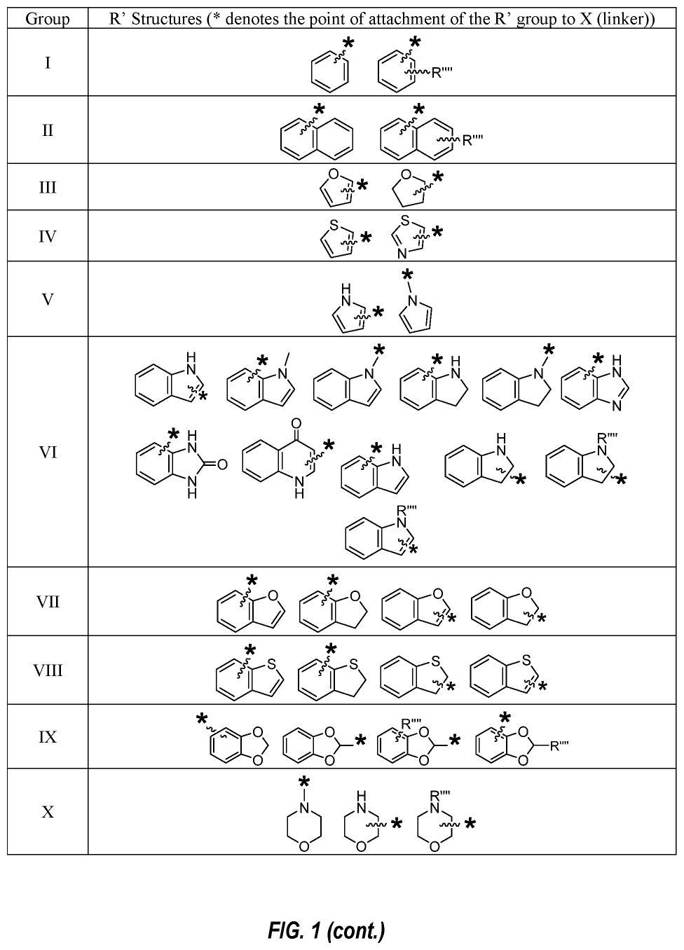

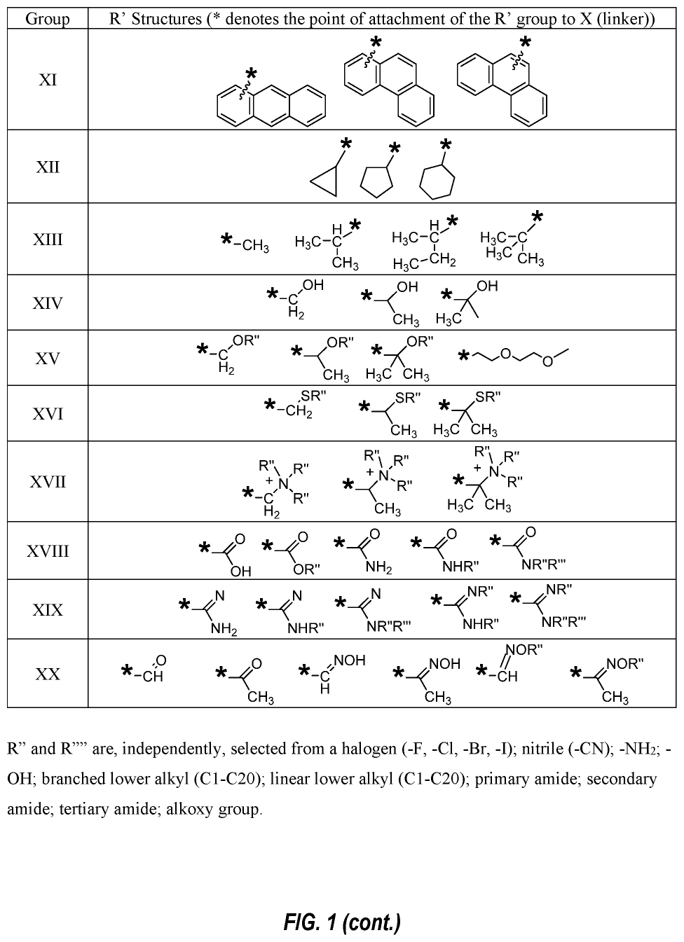

C-5 Modified Pyrimidine: As used herein, the term “C-5 modified pyrimidine” refers to a pyrimidine with a modification at the C-5 position including, but not limited to, those moieties illustrated in FIGS. 1 to 3. Nonlimiting examples of a C-5 modified pyrimidine include those described in U.S. Pat. Nos. 5,719,273 and 5,945,527. Nonlimiting examples of a nucleoside comprising a C-5 modification include substitution of deoxyuridine at the C-5 position with a substituent independently selected from: benzylcarboxyamide (alternatively benzylaminocarbonyl) (Bn), naphthylmethylcarboxyamide (alternatively naphthylmethylaminocarbonyl) (Nap), tryptaminocarboxyamide (alternatively tryptaminocarbonyl) (Trp), phenethylcarboxyamide (alternatively phenethylamino carbonyl) (Pe), thiophenylmethylcarboxyamide (alternatively thiophenylmethylaminocarbonyl) (Th) and isobutylcarboxyamide (alternatively isobutylaminocarbonyl) (iBu) as illustrated herein.

Chemical modifications of a C-5 modified pyrimidine can also be combined with, singly or in any combination, other nucleoside modifications, such as 2′-position sugar modifications, modifications at exocyclic amines, and substitution of 4-thiouridine, etc.

Certain representative C-5 modified pyrimidine containing nucleosides include: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-isobutylcarboxyamide)-2′-deoxyuridine (iBudU), 5-(N-isobutylcarboxyamide)-2′-O-methyluridine, 5-(N-phenethylcarboxyamide)-2′-deoxyuridine (PedU), 5-(N-thiophenylmethylcarboxyamide)-2′-deoxyuridine (ThdU), 5-(N-isobutylcarboxyamide)-2′-fluorouridine, 5-(N-tryptaminocarboxyamide)-2′-deoxyuridine (TrpdU), 5-(N-tryptaminocarboxyamide)-2′-O-methyluridine, 5-(N-tryptaminocarboxyamide)-2′-fluorouridine, 5-(N-[1-(3-trimethylamonium) propyl] carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine or 5-(N-[1-(2,3-dihydroxypropyl)]carboxyamide)-2′-deoxyuridine).

Nucleotides can be modified either before or after synthesis of an oligonucleotide. A sequence of nucleotides in an oligonucleotide may be interrupted by one or more non-nucleotide components. A modified oligonucleotide may be further modified after polymerization, such as, for example, by conjugation with any suitable labeling component.

As used herein, the term “at least one pyrimidine,” when referring to modifications of a nucleic acid, refers to one, several, or all pyrimidines in the nucleic acid, indicating that any or all occurrences of any or all of C, T, or U in a nucleic acid may be modified or not.

IgG Aptamer: IgG aptamer, as used herein, refers to an aptamer that is capable of binding to a IgG protein, which includes total IgG, one or more of the individual subclasses (IgG1, IgG2, IgG3 and IgG4), an IgG Fc region, and IgG paired with a light chain constant region, such as a kappa light chain constant region or a lambda light chain constant region, which pairing may be in the context of an antibody. The IgG aptamer may exhibit specificity for each one of these subclass and/or regions, or may bind all or a subset of the subclasses and/or regions.

Consensus Sequence: Consensus sequence, as used herein, refers to a nucleobase sequence that represents the most frequently observed nucleotide found at each position of a series of nucleic acid sequences subject to a sequence alignment.

Inhibit: The term inhibit, as used herein, means to prevent or reduce the expression of a peptide or a polypeptide to an extent that the peptide or polypeptide no longer has measurable activity or bioactivity; or to reduce the stability and/or reduce or block the activity of a peptide or a polypeptide to an extent that the peptide or polypeptide no longer has measurable activity.

Modulate: The term modulate, as used herein, means to alter the expression level of a peptide, protein or polypeptide by increasing or decreasing its expression level relative to a reference expression level, and/or alter the stability and/or activity of a peptide, protein or polypeptide by increasing or decreasing its stability and/or activity level relative to a reference stability and/or activity level.

Pharmaceutically Acceptable Salt: Pharmaceutically acceptable salt or salt of a compound (e.g., aptamer), as used herein, refers to a product that contains an ionic bond and is typically produced by reacting the compound with either an acid or a base, suitable for administering to an individual. A pharmaceutically acceptable salt can include, but is not limited to, acid addition salts including hydrochlorides, hydrobromides, phosphates, sulphates, hydrogen sulphates, alkylsulphonates, arylsulphonates, arylalkylsulfonates, acetates, benzoates, citrates, maleates, fumarates, succinates, lactates, and tartrates; alkali metal cations such as Li, Na, K, alkali earth metal salts such as Mg or Ca, or organic amine salts.

Pharmaceutical Composition: Pharmaceutical composition, as used herein, refers to formulation comprising an aptamer in a form suitable for administration to an individual. A pharmaceutical composition is typically formulated to be compatible with its intended route of administration. Examples of routes of administration include, but are not limited to, oral and parenteral, e.g., intravenous, intradermal, subcutaneous, inhalation, topical, transdermal, transmucosal, and rectal administration.

SELEX: The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

Sequence Identity: Sequence identity, as used herein, in the context of two or more nucleic acid sequences is a function of the number of identical nucleobase positions shared by the sequences (i.e., % identity=number of identical positions/total number of positionsx 100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST). For sequence comparisons, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith and Waterman, Adv. Appl. Math., 2:482, 1981, by the homology alignment algorithm of Needleman and Wunsch, J. Mol. Biol., 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel, F. M. et al., Current Protocols in Molecular Biology, pub. by Greene Publishing Assoc. and Wiley-Interscience (1987)). As used herein, when describing the percent identity of a nucleic acid, such as an aptamer, the sequence of which is at least, for example, about 95% identical to a reference nucleobase sequence, it is intended that the nucleic acid sequence is identical to the reference sequence except that the nucleic acid sequence may include up to five point mutations per each 100 nucleotides of the reference nucleic acid sequence. In other words, to obtain a desired nucleic acid sequence, the sequence of which is at least about 95% identical to a reference nucleic acid sequence, up to 5% of the nucleobases in the reference sequence may be deleted or substituted with another nucleobase, or some number of nucleobases up to 5% of the total number of nucleobases in the reference sequence may be inserted into the reference sequence (referred to herein as an insertion). These mutations of the reference sequence to generate the desired sequence may occur at the 5′ or 3′ terminal positions of the reference nucleotide sequence or anywhere between those terminal positions, interspersed either individually among nucleobases in the reference sequence or in one or more contiguous groups within the reference sequence.

SOMAmer: As used herein, a “SOMAmer” or Slow Off-Rate Modified Aptamer, refers to an aptamer having improved off-rate characteristics. SOMAmers can be generated using the improved SELEX methods described in U.S. Pat. No. 7,947,447, entitled “Method for Generating Aptamers with Improved Off-Rates,” which is incorporated by reference in its entirety. In some embodiments, a slow off-rate aptamer (including an aptamers comprising at least one nucleotide with a hydrophobic modification) has an off-rate (t½) of ≥2 minutes, ≥4 minutes, ≥5 minutes, ≥8 minutes, ≥10 minutes, ≥15 minutes ≥30 minutes, ≥60 minutes, ≥90 minutes, ≥120 minutes, ≥150 minutes, ≥180 minutes, ≥210 minutes, or ≥240 minutes.

Spacer Sequence: Spacer sequence, as used herein, refers to any sequence comprised of small molecule(s) covalently bound to the 5′-end, 3′-end, both 5′ and 3′ ends and/or between nucleotides of the nucleic acid sequence of an aptamer. Exemplary spacer sequences include, but are not limited to, polyethylene glycols, hydrocarbon chains, and other polymers or copolymers that provide a molecular covalent scaffold connecting the consensus regions while preserving aptamer binding activity. In certain aspects, the spacer sequence may be covalently attached to the aptamer through standard linkages such as the terminal 3′ or 5′ hydroxyl, 2′ carbon, or base modification such as the C5-position of pyrimidines, or C8 position of purines.

Target Molecule: Target molecule (or target), as used herein, refers to any compound or molecule upon which a nucleic acid can act in a desirable manner (e.g., binding of the target, catalytically changing the target, reacting with the target in a way that modifies or alters the target or the functional activity of the target, covalently attaching to the target (as in a suicide inhibitor), and facilitating the reaction between the target and another molecule). Non-limiting examples of a target molecule include a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc. Virtually any chemical or biological effector may be a suitable target. Molecules of any size can serve as targets. A target can also be modified in certain ways to enhance the likelihood or strength of an interaction between the target and the nucleic acid. A target may also include any minor variation of a particular compound or molecule, such as, in the case of a protein, for example, variations in its amino acid sequence, disulfide bond formation, glycosylation, lipidation, acetylation, phosphorylation, or any other manipulation or modification, such as conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules.

The singular terms “a,” “an,” and “the” include plural referents unless context clearly indicates otherwise. It should be understood that the terms “a” and “an” as used herein refer to “one or more” of the enumerated components. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” means ±20% of the indicated range, value, or structure, unless otherwise indicated. As used herein, the terms “include” and “comprise” are open ended and are used synonymously.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### SELEX

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5- and 2-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication 20090098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Patent Application Publication 20090004667, entitled “Method for Generating Aptamers with Improved Off-Rates”, which describes improved SELEX methods for generating aptamers that can bind to target molecules. As mentioned above, these slow off-rate aptamers are known as “SOMAmers.” Methods for producing aptamers or SOMAmers and photoaptamers or SOMAmers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers or SOMAmers with improved off-rate performance.

A variation of this assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers.

In both of these assay formats, the aptamers or SOMAmers are immobilized on the solid support prior to being contacted with the sample. Under certain circumstances, however, immobilization of the aptamers or SOMAmers prior to contact with the sample may not provide an optimal assay. For example, pre-immobilization of the aptamers or SOMAmers may result in inefficient mixing of the aptamers or SOMAmers with the target molecules on the surface of the solid support, perhaps leading to lengthy reaction times and, therefore, extended incubation periods to permit efficient binding of the aptamers or SOMAmers to their target molecules. Further, when photoaptamers or photoSOMAmers are employed in the assay and depending upon the material utilized as a solid support, the solid support may tend to scatter or absorb the light used to effect the formation of covalent bonds between the photoaptamers or photoSOMAmers and their target molecules. Moreover, depending upon the method employed, detection of target molecules bound to their aptamers or photoSOMAmers can be subject to imprecision, since the surface of the solid support may also be exposed to and affected by any labeling agents that are used. Finally, immobilization of the aptamers or SOMAmers on the solid support generally involves an aptamer or SOMAmer-preparation step (i.e., the immobilization) prior to exposure of the aptamers or SOMAmers to the sample, and this preparation step may affect the activity or functionality of the aptamers or SOMAmers.

SOMAmer assays that permit a SOMAmer to capture its target in solution and then employ separation steps that are designed to remove specific components of the SOMAmer-target mixture prior to detection have also been described (see U.S. Patent Application Publication 20090042206, entitled “Multiplexed Analyses of Test Samples”). The described SOMAmer assay methods enable the detection and quantification of a non-nucleic acid target (e.g., a protein target) in a test sample by detecting and quantifying a nucleic acid (i.e., a SOMAmer). The described methods create a nucleic acid surrogate (i.e., the SOMAmer) for detecting and quantifying a non-nucleic acid target, thus allowing the wide variety of nucleic acid technologies, including amplification, to be applied to a broader range of desired targets, including protein targets.

Embodiments of the SELEX process in which the target is a peptide are described in U.S. Pat. No. 6,376,190, entitled “Modified SELEX Processes Without Purified Protein.” In the instant case, the target is the IgG protein.

### Chemically Modified Aptamers

Aptamers may contain modified nucleotides that improve their properties and characteristics. Non-limiting examples of such improvements include in vivo stability, stability against degradation, binding affinity for its target, and/or improved delivery characteristics.

Examples of such modifications include chemical substitutions at the ribose and/or phosphate and/or base positions of a nucleotide. SELEX process-identified aptamers containing modified nucleotides are described in U.S. Pat. No. 5,660,985, entitled “High Affinity Nucleic Acid Ligands Containing Modified Nucleotides,” which describes oligonucleotides containing nucleotide derivatives chemically modified at the 5- and 2-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication No. 20090098549, entitled “SELEX and PHOTOSELEX,” which describes nucleic acid libraries having expanded physical and chemical properties and their use in SELEX and photoSELEX.

As used herein, the term “nucleotide” refers to a ribonucleotide or a deoxyribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs). As used herein, the term “cytidine” is used generically to refer to a ribonucleoside, deoxyribonucleoside, or modified ribonucleoside comprising a cytosine base, unless specifically indicated otherwise. The term “cytidine” includes 2′-modified cytidines, such as 2′-fluoro, 2′-methoxy, etc. Similarly, the term “modified cytidine” or a specific modified cytidine also refers to a ribonucleoside, deoxyribonucleoside, or modified ribonucleoside (such as 2′-fluoro, 2′-methoxy, etc.) comprising a modified cytosine base, unless specifically indicated otherwise. The term “uridine” is used generically to refer to a ribonucleoside, deoxyribonucleoside, or modified ribonucleoside comprising a uracil base, unless specifically indicated otherwise. The term “uridine” includes 2′-modified uridines, such as 2′-fluoro, 2′-methoxy, etc. Similarly, the term “modified uridine” or a specific modified uridine also refers to a ribonucleoside, deoxyribonucleoside, or modified ribonucleoside (such as 2′-fluoro, 2′-methoxy, etc.) comprising a modified uracil base, unless specifically indicated otherwise.

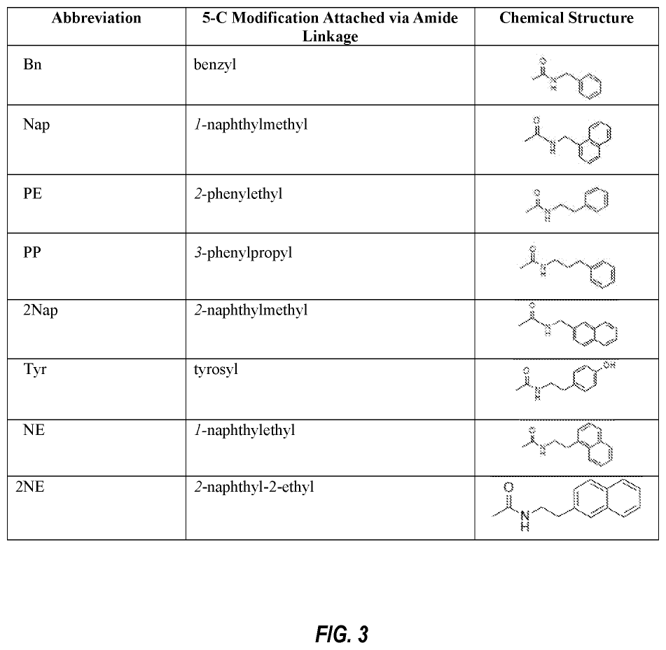

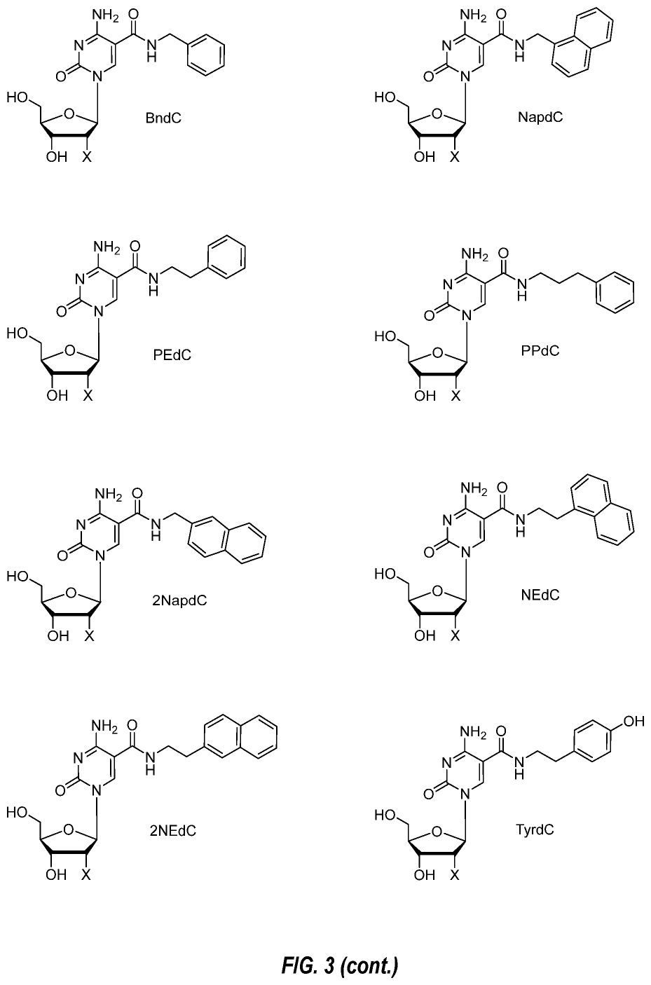

As used herein, the term “5-position modified cytidine” or “C-5 modified cytidine” refers to a cytidine with a modification at the C-5 position of the cytosine base. As used herein, the term “C-5 modified carboxamidecytidine” or “cytidine-5-carboxamide” refers to a cytidine with a carboxyamide (—C(O)NH—) modification at the C-5 position of the cytosine base including, but not limited to, those moieties (R^{X1}) illustrated herein. Exemplary C-5 modified carboxamidecytidines include, but are not limited to, 5-(N-benzylcarboxamide)-2′-deoxycytidine (referred to as “BndC” and shown in FIG. 3); 5-(N-2-phenylethylcarboxamide)-2′-deoxycytidine (referred to as “PEdC” and shown in FIG. 3); 5-(N-3-phenylpropylcarboxamide)-2′-deoxycytidine (referred to as “PPdC” and shown in FIG. 3); 5-(N-1-naphthylmethylcarboxamide)-2′-deoxycytidine (referred to as “NapdC” and shown in FIG. 3); 5-(N-2-naphthylmethylcarboxamide)-2′-deoxycytidine (referred to as “2NapdC” and shown in FIG. 3); 5-(N-1-naphthyl-2-ethylcarboxamide)-2′-deoxycytidine (referred to as “NEdC” and shown in FIG. 3); 5-(N-2-naphthyl-2-ethylcarboxamide)-2′-deoxycytidine (referred to as “2NEdC” and shown in FIG. 3); and 5-(N-tyrosylcarboxyamide)-2′-deoxycytidine (referred to as TyrdC and shown in FIG. 3). In some embodiments, the C5-modified cytidines, e.g., in their triphosphate form, are capable of being incorporated into an oligonucleotide by a polymerase (e.g., KOD DNA polymerase).

Chemical modifications of the C-5 modified cytidines described herein can also be combined with, singly or in any combination, 2′-position sugar modifications, modifications at exocyclic amines, and substitution of 4-thiocytidine and the like.

As used herein, the term “5-position modified cytosine” or “C-5 modified cytosine” refers to a cytosine base with a modification at the C-5 position of the cytosine. As used herein, the term “C-5 modified carboxamidecytosine” or “cytosine-5-carboxamide” refers to a cytosine base with a carboxyamide (—C(O)NH—) modification at the C-5 position of the cytosine including, but not limited to, those moieties (R^{X1}) illustrated herein. Exemplary C-5 modified carboxamidecytosines include, but are not limited to, the modified cytosines shown in FIG. 3.

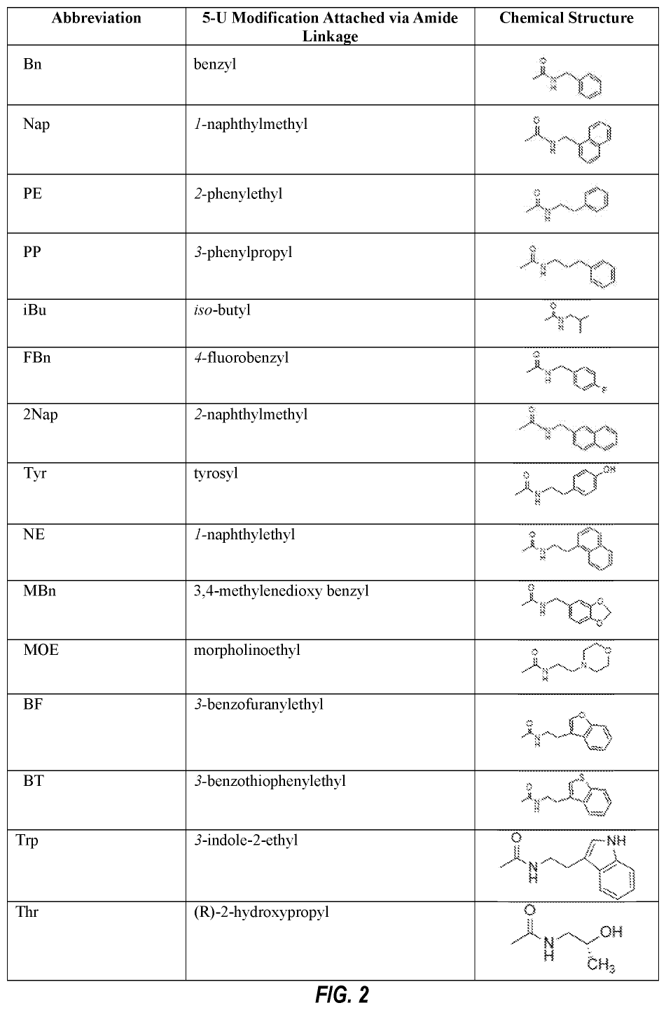

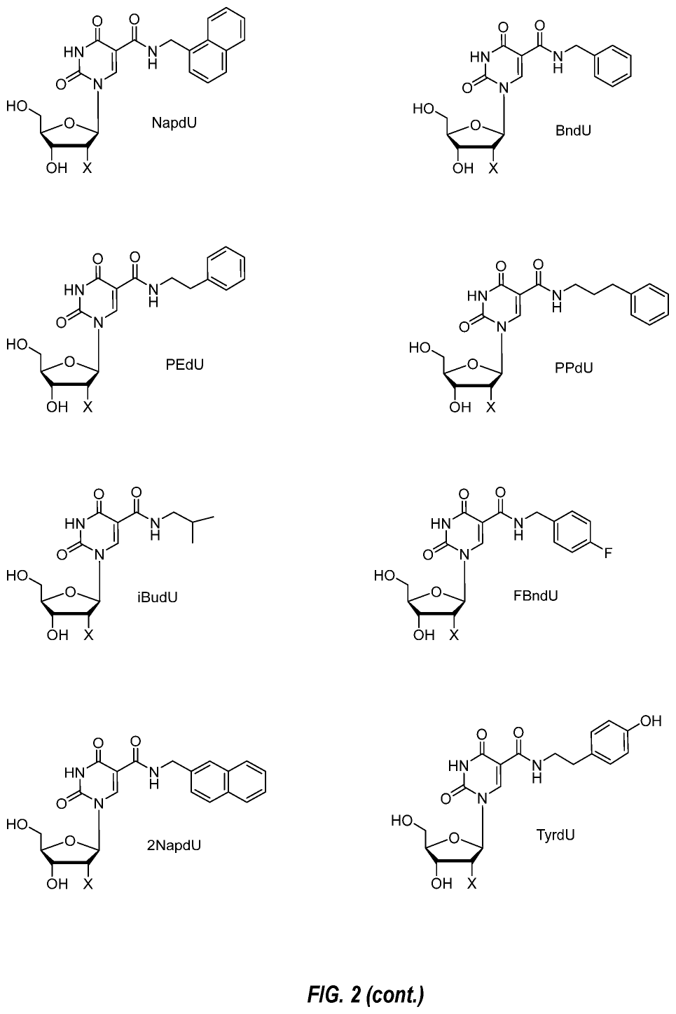

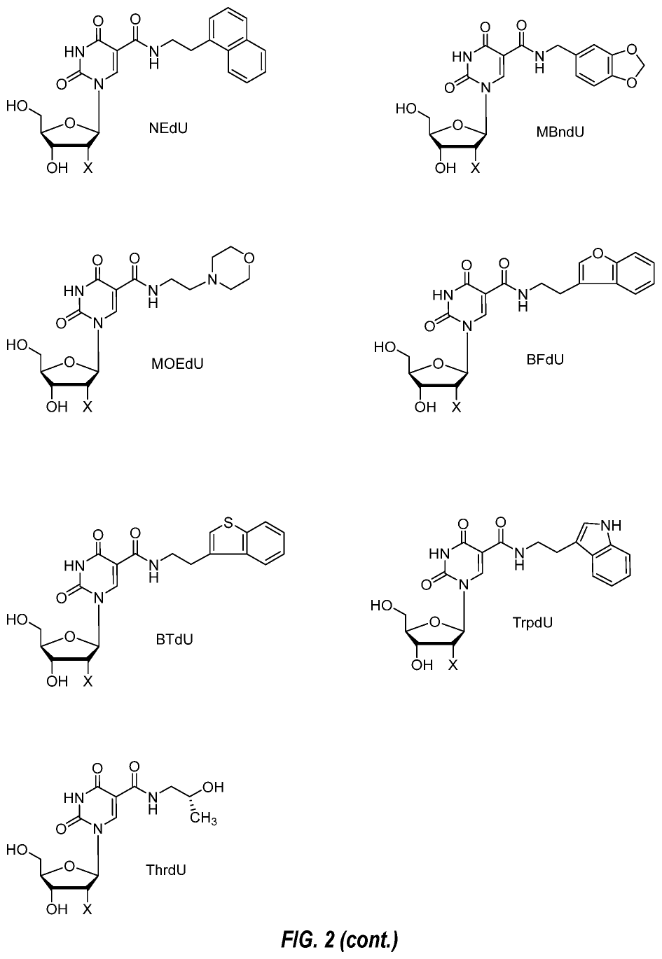

As used herein, the term “C-5 modified uridine” or “5-position modified uridine” refers to a uridine or a deoxyuridine with modification at the C-5 position of the uracil base. In some embodiments, a uridine or a deoxyuridine has a carboxyamide (—C(O)NH—) modification at the C-5 position of the uracil base, e.g., as shown in FIG. 2. In some embodiments, the C5-modified uridines, e.g., in their triphosphate form, are capable of being incorporated into an oligonucleotide by a polymerase (e.g., KOD DNA polymerase). Nonlimiting exemplary 5-position modified uridines include:
5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N—R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

As used herein, the terms “modify,” “modified,” “modification,” and any variations thereof, when used in reference to an oligonucleotide, means that at least one of the nucleotide bases (such as an A, G, T/U, and/or C) of the oligonucleotide is an analog or ester of a naturally occurring nucleotide. In some embodiments, the modified nucleotide has greater nuclease resistance than the unmodified oligonucleotide. Additional modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleoside modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers.

As used herein, a “hydrophobic group” and “hydrophobic moiety” are used interchangeably herein and refer to any group or moiety that is uncharged and/or has a small dipole and/or the group or moiety tends to repel from water. These groups or moieties may comprise, for example, an aromatic hydrocarbon or a planar aromatic hydrocarbon. Methods for determining the hydrophobicity or whether molecule (or group or moiety) is hydrophobic are well known in the art and include empirically derived methods, as well as calculation methods. Exemplary methods are described in Zhu Chongqin et al. (2016) Characterizing hydrophobicity of amino acid side chains in a protein environment via measuring contact angle of a water nanodroplet on planar peptide network. Proc. Natl. Acad. Sci., 113(46) pgs. 12946-12951. As disclosed herein, exemplary hydrophobic moieties included, but are not limited to, Groups I, II, III, IV, V, VII, VIII, IX, XI, XII, XIII, XV and XVI of FIG. 1. Further exemplary hydrophobic moieties include those of FIG. 2 (e.g., Bn, Nap, PE, PP, iBu, 2Nap, Try, NE, MBn, BF, BT, Trp).

As used herein, “protein” is used synonymously with “peptide” and “polypeptide”. A “purified” polypeptide, protein, or peptide is substantially free of cellular material or other contaminating proteins from the cell, tissue, or cell-free source from which the amino acid sequence is obtained, or substantially free from chemical precursors or other chemicals when chemically synthesized.

As used herein, the term “nucleic acid” refers to any nucleic acid sequence containing DNA and/or RNA and/or analogs thereof and includes single, double and multi-stranded forms. As used herein, the terms “nucleic acid,” “oligo,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers.

Polynucleotides can also contain analogous forms of ribose or deoxyribose sugars that are generally known in the art, including 2′-O-methyl, 2′-O-allyl, 2′-O-ethyl, 2′-O-propyl, 2′-O—CH₂CH₂OCH₃, 2′-fluoro, 2′-NH2 or 2′-azido, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted herein, one or more phosphodiester linkages may be replaced by alternative linking groups. These alternative linking groups include embodiments wherein phosphate is replaced by phosphorothioate, P(O)S (“thioate”), P(S)S (“dithioate”), (O)NR^{X}₂ (“amidate”), P(O) R^{X}, P(O)OR^{X}′, CO or CH₂ (“formacetal”), in which each R^{X} or R′ are independently H or substituted or unsubstituted alkyl (C1-C20) optionally containing an ether (—O—) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

If present, a modification to the nucleotide structure can be imparted before or after assembly of a polymer. A sequence of nucleotides can be interrupted by non-nucleotide components. A polynucleotide can be further modified after polymerization, such as by conjugation with a labeling component.

As used herein, the term “at least one nucleotide” when referring to modifications of a nucleic acid, refers to one, several, or all nucleotides in the nucleic acid, indicating that any or all occurrences of any or all of A, C, T, G or U in a nucleic acid may be modified or not.

As used herein, an aptamer comprising a single type of 5-position modified pyrimidine or C-5 modified pyrimidine may be referred to as “single modified aptamers”, aptamers having a “single modified base”, aptamers having a “single base modification” or “single bases modified”, all of which may be used interchangeably. A library of aptamers or aptamer library may also use the same terminology.

As used herein, an aptamer comprising two different types of 5-position modified pyrimidines (or C-5 modified pyrimidines) may be referred to as “dual modified aptamers”, aptamers having “two modified bases”, aptamers having “two base modifications” or “two bases modified”, aptamer having “double modified bases”, all of which may be used interchangeably. A library of aptamers or aptamer library may also use the same terminology. Thus, in some embodiments, an aptamer comprises two different 5-position modified pyrimidines wherein the nucleosides comprising the two different 5-position modified pyrimidines are selected from a NapdC and a NapdU, a NapdC and a PPdU, a NapdC and a MOEdU, a NapdC and a TyrdU, a NapdC and a ThrdU, a PPdC and a PPdU, a PPdC and a NapdU, a PPdC and a MOEdU, a PPdC and a TyrdU, a PPdC and a ThrdU, a NapdC and a 2NapdU, a NapdC and a TrpdU, a 2NapdC and a NapdU, and 2NapdC and a 2NapdU, a 2NapdC and a PPdU, a 2NapdC and a TrpdU, a 2NapdC and a TyrdU, a PPdC and a 2NapdU, a PPdC and a TrpdU, a PPdC and a TyrdU, a TyrdC and a TyrdU, a TrydC and a 2NapdU, a TyrdC and a PPdU, a TyrdC and a TrpdU, a TyrdC and a TyrdU, and a TyrdC and a TyrdU. In some embodiments, an aptamer comprises at least one modified uridine and/or thymidine and at least one modified cytidine, wherein the at least one modified uridine and/or thymidine is modified at the 5-position with a moiety selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety, and wherein the at least one modified cytidine is modified at the 5-position with a moiety selected from a naphthyl moiety, a tyrosyl moiety, and a benzyl moiety. In certain embodiments, the moiety is covalently linked to the 5-position of the base via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.

In certain embodiments, an aptamer comprises a first 5-position modified pyrimidine and a second 5-position modified pyrimidine, wherein the first 5-position modified pyrimidine comprises a tryosyl moiety at the 5-position of the first 5-position modified pyrimidine, and the second 5-position modified pyrimidine comprises a naphthyl moiety or benzyl moiety at the 5-position at the second 5-position modified pyrimidine. In a related embodiment the first 5-position modified pyrimidine is a uracil. In a related embodiment, the second 5-position modified pyrimidine is a cytosine. In a related embodiment, at least 10%, 15%, 20%, 25%, 30%, 35%, 40%, 45%, 50%, 55%, 60%, 65%, 70%, 75%, 80%, 85%, 90%, 95%, or 100% of the uracils of the aptamer are modified at the 5-position. In a related embodiment, at least 10%, 15%, 20%, 25%, 30%, 35%, 40%, 45%, 50%, 55%, 60%, 65%, 70%, 75%, 80%, 85%, 90%, 95%, or 100% of the cytosine of the aptamer are modified at the 5-position.

### Exemplary NapdU Structure (5-[N-(1-naphthylmethyl)carboxamide]-2′-deoxyuridine)

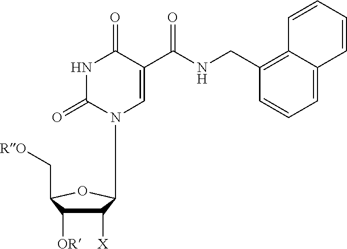

### Exemplary 2NapdU structure (5-[N-(2-naphthylmethyl)carboxamide]-2′-deoxyuridine)

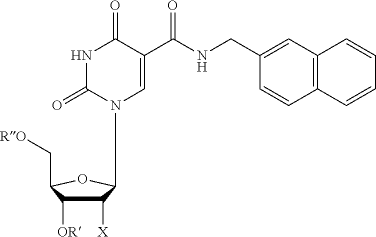

### Exemplary PPdU structure (5[N-(phenyl-3-propyl)carboxamide]-2′-deoxyuridine)

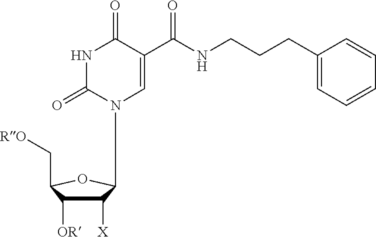

### Exemplary TrpdU structure (5-[N-(3-indole-2-ethyl)carboxamide]-2′-deoxyuridine)

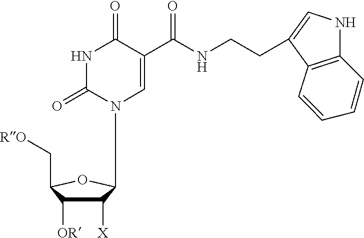

### Exemplary 2NEdU structure 5-[N-(2-naphthyl-2-ethyl)carboxamide]-2′-deoxyuridine)

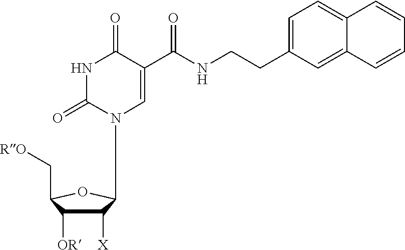

### Methods of Detecting IgG

In some embodiments, methods of detecting IgG in a sample are provided, comprising contacting the sample with an aptamer described herein. In some embodiments, methods of detecting or quantifying IgG are provided, comprising contacting a sample that contains an IgG or is suspected of containing an IgG with aptamer described herein. In some embodiments, methods of distinguishing IgG1, IgG2, IgG3, and/or IgG4 from one another in a sample are provided, comprising contacting the sample with an aptamer described herein. In some embodiments, the method comprises contacting the sample with an IgG aptamer described herein in the presence of a polyanionic inhibitor. Detecting and/or quantifying IgG bound by the IgG aptamer can be accomplished using methods in the art and/or methods described herein. In some embodiments, the IgG aptamer comprises a detectable label. In some embodiments, the IgG aptamer is bound to a solid support, or comprises a member of a binding pair that may be captured on a solid support (for example, a biotinylated aptamer may be bound to a solid support comprising streptavidin).

### Kits Comprising IgG Aptamer Compositions

The present disclosure provides kits comprising any of the IgG aptamers described herein. Such kits can comprise, for example, (1) at least one IgG aptamer; and (2) at least one solid support. Additional kit components can optionally include, for example: (1) any stabilizers, buffers, etc., and (2) at least one container, vial or similar apparatus for holding and/or mixing the kit components.

The following examples are provided to illustrate certain particular features and/or embodiments. These examples should not be construed to limit the disclosure to the particular features or embodiments described.

### EXAMPLES

### Example 1

Two approaches for identifying aptamers that bind IgG were used. First, existing aptamer sequences raised to (IgG) Fc fusion proteins were screened for aptamers that bound to the Fc portion of the Fc fusion protein. To mine for the IgG binders, the aptamer sequences identified using SELEX for aptamers that bind to different Fc fusion proteins were aligned to identify common sequence patterns across the aptamer sequence databases. The common sequence patterns in each of the individual target proteins could be Fc IgG binders as typically, different protein targets result in different aptamer sequences (i.e., the commonality in sequences is the likely result of the presence of the Fc IgG fusion region).

A second related approach used known Fc IgG binders to search the SomaLogic aptamer sequence database to identify common sequences or sequences motifs.

The results of both approaches were combined to further identify common sequences. Three sequence patterns were identified and further explored for binding affinity to IgG. Based on the binding affinities, three aptamer sequences were further analyzed for binding affinity to IgG and subject to a truncation analysis to identify minimal sequence IgG binders.

### Example 2

The IgG binding affinities (or dissociation constant; K_{d}) for full length 50-mer sequences and truncated sequences of the 5406-56_3; 5334-8_3 and 14125-144_3 aptamer families were determined, and used to identify a minimal sequence length that is capable of binding to IgG for each aptamer family.

Briefly, the dissociation constant (K_{d}) was measured for each aptamer using either Protein L or Zorbax bead partitioning. For Protein L assays, radiolabeled aptamer was renatured by heating to 95° C. for 3 minutes in SB17 (40 mM HEPES, 102 mM NaCl, 1 mM EDTA, 5 mM MgCl2, 5 mM KCl) and slowly cooling to 37° C. Aptamer-target protein complexes were formed by mixing approximately 40 pM of aptamer with a range of concentrations of target protein (final top concentration of either 500 nM or 100 nM) in SB17, and incubating at 37° C. One-twelfth of each reaction was transferred to a nylon membrane and dried to determine total counts in each reaction. 55 μg of Protein L magnetic beads (Pierce) and 55 μL of 10 mM DxSO4 (dextran sulfate) was added to the remainder of each reaction and mixed at 37° C. for five minutes. Two-thirds of the reaction was then passed through a MultiScreen HV Plate (Millipore) under vacuum to separate protein-bound complexes from unbound aptamer and washed with 100 μL SB17. The nylon membrane and MultiScreen HV Plates were phosphorimaged and the amount of radioactivity in each sample quantified using a Typhoon FLA 7000 IP. The fraction of captured aptamer was plotted as a function of protein concentration and a non-linear curve-fitting algorithm was used to determine the dissociation constants (or K_{d} values) from the data. IgG1, 2, 3 and 4; Kappa and IgG1 Mouse proteins were measured using Protein L beads. All other proteins were measured using Zorbax beads.

For Zorbax assays radiolabeled aptamer was renatured by heating to 95° C. for 3 minutes in SB18 and slowly cooling to 37° C. Aptamer-target protein complexes were formed by mixing approximately 40 pM of aptamer with a range of concentrations of target protein (final top concentration of either 500 nM or 100 nM) in SB18 (40 mM HEPES, pH 7.5, 105 mM NaCl, 5 mM KCl, 5 mM MgCl2), and incubating at 37° C. One-twelfth of each reaction was transferred to a nylon membrane and dried to determine total counts in each reaction. 2.2 μg of Zorbax beads (Agilent) was added to the remainder of each reaction. Two-thirds of the reaction was then passed through a MultiScreen HV Plate (Millipore) under vacuum to separate protein-bound complexes from unbound aptamer and washed with 185 μL SB18. The nylon membrane and MultiScreen HV Plates were phosphorimaged and the amount of radioactivity in each sample quantified using a Typhoon FLA 7000 IP. The fraction of captured aptamer was plotted as a function of protein concentration and a non-linear curve-fitting algorithm was used to determine dissociation constants (or K_{d} values) from the data.

Table 1 shows the K_{d} values for the 5406-56_3 (50-mer; SEQ ID NO: 1) aptamer for IgG, and the 5′-end and 3′-end truncation analysis of the 50-mer. For Table 1, “P” in each sequence represents a NapdU. The sequences in Table 1 are aligned to show how each truncated sequence overlaps with the parent 50-mer sequence (5406-56_3).

TABLE 1
5406-56 aptamer truncation series (5' or 3' truncations)

| | Kd | AptamerNucleic Acid Sequence (5' to 3') | SEQ ID |
|---|---|---|---|
| Aptamer ID | (nM) | (P is a NapdU) | NO: |
| 5406-56_3 | 3.13 | CCTCCAPACGGPACGAPPPPCAGPPGGAPCCAGPACCPGGCPGGAGAGAA | 1 |
| 5406-56_4 | 3.11 | TCCAPACGGPACGAPPPPCAGPPGGAPCCAGPACCPGGCPGGAGAGAA | 2 |
| 5406-56_5 | 7.91 | CAPACGGPACGAPPPPCAGPPGGAPCCAGPACCPGGCPGGAGAGAA | 3 |
| 5406-56_6 | 9.05 | PACGGPACGAPPPPCAGPPGGAPCCAGPACCPGGCPGGAGAGAA | 4 |
| 5406-56_7 | 13 | CGGPACGAPPPPCAGPPGGAPCCAGPACCPGGCPGGAGAGAA | 5 |
| 5406-56_8 | 78.1 | GPACGAPPPPCAGPPGGAPCCAGPACCPGGCPGGAGAGAA | 6 |
| 5406-56_9 | >1000 | ACGAPPPPCAGPPGGAPCCAGPACCPGGCPGGAGAGAA | 7 |
| 5406-56_10 | >1000 | GAPPPPCAGPPGGAPCCAGPACCPGGCPGGAGAGAA | 8 |
| 5406-56_11 | >1000 | PPPPCAGPPGGAPCCAGPACCPGGCPGGAGAGAA | 9 |
| 5406-56_12 | 2.88 | CCTCCAPACGGPACGAPPPPCAGPPGGAPCCAGPACCPGGCPGGAGAG | 10 |
| 5406-56_13 | 2.61 | CCTCCAPACGGPACGAPPPPCAGPPGGAPCCAGPACCPGGCPGGAG | 11 |
| 5406-56_14 | 3 | CCTCCAPACGGPACGAPPPPCAGPPGGAPCCAGPACCPGGCPGG | 12 |
| 5406-56_15 | 4.64 | CCTCCAPACGGPACGAPPPPCAGPPGGAPCCAGPACCPGGCP | 13 |
| 5406-56_16 | 2.58 | CCTCCAPACGGPACGAPPPPCAGPPGGAPCCAGPACCPGG | 14 |
| 5406-56_17 | 4.14 | CCTCCAPACGGPACGAPPPPCAGPPGGAPCCAGPACCP | 15 |
| 5406-56_18 | 42.2 | CCTCCAPACGGPACGAPPPPCAGPPGGAPCCAGPAC | 16 |
| 5406-56_19 | >1000 | CCTCCAPACGGPACGAPPPPCAGPPGGAPCCAGP | 17 |

The data from Table 1 shows that SEQ ID Nos: 1, 2, 3, 4, 5, 6, 10, 11, 12, 13, 14, 15 and 16 have K_{d} values from about 2.5 nM to about 78 nM indicating that certain 5′-end nucleotides of the aptamer may be removed, and separately, that certain 3′-end nucleotides of the aptamer may be removed, and the aptamer retains binding capability to IgG. The data from Table 1 also indicates that the removal of more than 12 nucleotides from the 5′-end of 5406-56_3 (see SEQ ID NOs: 7, 8 and 9), and removal of more than 16 nucleotide from the 3-end of 5406-56_3 (see SEQ ID NOs: 17), results in K_{d} values of greater than 1000 nM (or >1000 nM), which is considered to be a “no binding” (or NB) result for the dissociation constant assay.

To further understand the contribution of the 5′-end and 3′-end nucleotides of the 5406-56_3 (SEQ ID NO:1) aptamer to IgG binding, additional truncations were generated with both 5′ and 3′end nucleotides were removed. The K_{d} values for each aptamer is shown in Table 2. For table 2, “P” in each sequence represents a NapdU. The sequences in Table 2 are aligned to show how each truncated sequence overlaps with the parent 50-mer sequence (5406-56_3).

TABLE 2
5406-56 aptamer 5'-end and 3'-end truncation series

| Aptamer ID | Kd | Aptamer Nucleic Acid Sequence (5' to 3') | SEQ ID |
|---|---|---|---|
| (NapdU) | (nM) | (P is a NapdU) | NO: |
| 5406-56_3 | 4.54 | CCTCCAPACGGPACGAPPPPCAGPPGGAPCCAGPACCPGGCPGGAGAGAA | 1 |
| 5406-56_21 | 8.13 | GPACGAPPPPCAGPPGGAPCCAGPACCPGG | 18 |
| 5406-56_22 | 4.93 | GPACGAPPPPCAGPPGGAPCCAGPACCPGG | 19 |
| 5406-56_23 | 5.48 | GPACGAPPPPCAGPPGGAPCCAGPACCPGG | 20 |
| 5406-56_24 | 5.1 | GPACGAPPPPCAGPPGGAPCCAGPACCPGG | 21 |
| 5406-56_25 | 21.9 | GPACGAPPPPCAGPPGGAPCCAGPACCPGG | 22 |
| 5406-56_26 | 64.3 | PACGAPPPPCAGPPGGAPCCAGPACCPGG | 23 |
| 5406-56_27 | 5.56 | PACGGPACGAPPPPCAGPPGGAPCCAGPACCPG | 24 |
| 5406-56_28 | 3.64 | ACGGPACGAPPPPCAGPPGGAPCCAGPACCPG | 25 |
| 5406-56_29 | 3.49 | CGGPACGAPPPPCAGPPGGAPCCAGPACCPG | 26 |
| 5406-56_30 | 3.22 | GGPACGAPPPPCAGPPGGAPCCAGPACCPG | 27 |
| 5406-56_31 | 14.8 | GPACGAPPPPCAGPPGGAPCCAGPACCPG | 28 |
| 5406-56_32 | 75.4 | PACGAPPPPCAGPPGGAPCCAGPACCPG | 29 |
| 5406-56_33 | 5.31 | PACGGPACGAPPPPCAGPPGGAPCCAGPACCP | 30 |
| 5406-56_34 | 3.36 | ACGGPACGAPPPPCAGPPGGAPCCAGPACCP | 31 |
| 5406-56_35 | 3.42 | CGGPACGAPPPPCAGPPGGAPCCAGPACCP | 32 |
| 5406-56_36 | 3.44 | GGPACGAPPPPCAGPPGGAPCCAGPACCP | 33 |
| 5406-56_37 | 9.44 | GPACGAPPPPCAGPPGGAPCCAGPACCP | 34 |
| 5406-56_38 | >1000 | PACGAPPPPCAGPPGGAPCCAGPACCP | 35 |
| 5406-56_39 | 6.74 | PACGGPACGAPPPPCAGPPGGAPCCAGPACC | 36 |
| 5406-56_40 | 4.32 | ACGGPACGAPPPPCAGPPGGAPCCAGPACC | 37 |
| 5406-56_41 | 2.74 | CGGPACGAPPPPCAGPPGGAPCCAGPACC | 38 |
| 5406-56_42 | 4.97 | GGPACGAPPPPCAGPPGGAPCCAGPACC | 39 |
| 5406-56_43 | 11 | GPACGAPPPPCAGPPGGAPCCAGPACC | 40 |
| 5406-56_44 | 36.2 | PACGAPPPPCAGPPGGAPCCAGPACC | 41 |
| 5406-56_45 | 21.8 | PACGGPACGAPPPPCAGPPGGAPCCAGPAC | 42 |
| 5406-56_46 | 8.14 | ACGGPACGAPPPPCAGPPGGAPCCAGPAC | 43 |
| 5406-56_47 | 5.7 | CGGPACGAPPPPCAGPPGGAPCCAGPAC | 44 |
| 5406-56_48 | 5.81 | GGPACGAPPPPCAGPPGGAPCCAGPAC | 45 |
| 5406-56_49 | 17.6 | GPACGAPPPPCAGPPGGAPCCAGPAC | 46 |
| 5406-56_50 | 18.8 | PACGAPPPPCAGPPGGAPCCAGPAC | 47 |

The data from table 2 shows that SEQ ID Nos: 18, 19, 20, 21, 22, 23, 24, 25, 26, 27, 28, 29, 30, 31, 32, 33, 34, 36, 37, 38, 39, 40, 41, 42, 43, 44, 45, 46 and 47 have K_{d} values from about 2.7 nM to about 64 nM indicating that 5′-end and 3′-end nucleotides of the aptamer may be removed, and the aptamer retains binding capability to IgG. The data from Table 2 also indicates that a 25-mer sequence (5406-56_50; SEQ ID NO: 47) is sufficient to bind IgG (K_{d} value of 18.8 nM). The following sequence is a “core” sequence sufficient to bind IgG (P is NapdU):

(SEQ ID NO: 47)
5'- PACGAPPPPCAGPPGGAPCCAGPAC -3'.

Thus, an aptamer that comprises additional nucleotides on the 5′-end and/or the 3′-end of SEQ ID NO:47 is expected to retain the ability to bind IgG as shown by the K_{d} values provided in Tables 1 and 2.

Additional “core” sequences sufficient to bind IgG include (P is NapdU):

(SEQ ID NO: 46)
5' - GPACGAPPPPCAGPPGGAPCCAGPAC - 3';
and
(SEQ ID NO: 45)
5' - GGPACGAPPPPCAGPPGGAPCCAGPAC - 3';

Thus, an aptamer that comprises additional nucleotides on the 5′-end and/or the 3′-end of SEQ ID NO:45 or 46, is expected to retain the ability to bind IgG as shown by the K_{d} values provided in Tables 1 and 2.

Table 3 shows the K_{d} values for the 5334-8_3 (50-mer; SEQ ID NO: 48) aptamer for IgG, and the 5′-end and 3′-end truncation analysis of the 50-mer. For Table 3, “P” in each sequence represents a 2NapdU. The sequences in Table 3 are aligned to show how each truncated sequence overlaps with the parent 50-mer sequence (5334-8_3).

TABLE 3
5334-8 aptamer truncation series (5' or 3' truncations)

| Aptamer | Kd | Aptamer Nucleic Acid Sequence (5' to 3') | SEQ ID |
|---|---|---|---|
| ID | (nM) | (P is a 2NapdU) | NO: |
| 5334-8_3 | 7.52 | CGGCACPCCACAPCCAAGACPGCGGPPACCPPGPAGGCGAPPAPGGGCAG | 48 |
| 5334-8_4 | 17.3 | GCACPCCACAPCCAAGACPGCGGPPACCPPGPAGGCGAPPAPGGGCAG | 49 |
| 5334-8_5 | 5.92 | ACPCCACAPCCAAGACPGCGGPPACCPPGPAGGCGAPPAPGGGCAG | 50 |
| 5334-8_6 | 7.89 | PCCACAPCCAAGACPGCGGPPACCPPGPAGGCGAPPAPGGGCAG | 51 |
| 5334-8_7 | 5.89 | CACAPCCAAGACPGCGGPPACCPPGPAGGCGAPPAPGGGCAG | 52 |
| 5334-8_8 | 9.69 | CAPCCAAGACPGCGGPPACCPPGPAGGCGAPPAPGGGCAG | 53 |
| 5334-8_9 | 8.38 | PCCAAGACPGCGGPPACCPPGPAGGCGAPPAPGGGCAG | 54 |
| 5334-8_10 | 5.38 | CAAGACPGCGGPPACCPPGPAGGCGAPPAPGGGCAG | 55 |
| 5334-8_11 | 4.69 | AGACPGCGGPPACCPPGPAGGCGAPPAPGGGCAG | 56 |
| 5334-8_12 | 25.8 | CGGCACPCCACAPCCAAGACPGCGGPPACCPPGPAGGCGAPPAPGGGC | 57 |
| 5334-8_13 | >1000 | CGGCACPCCACAPCCAAGACPGCGGPPACCPPGPAGGCGAPPAPGG | 58 |
| 5334-8_14 | >1000 | CGGCACPCCACAPCCAAGACPGCGGPPACCPPGPAGGCGAPPAP | 59 |
| 5334-8_15 | >1000 | CGGCACPCCACAPCCAAGACPGCGGPPACCPPGPAGGCGAPP | 60 |
| 5334-8_16 | >1000 | CGGCACPCCACAPCCAAGACPGCGGPPACCPPGPAGGCGA | 61 |
| 5334-8_17 | >1000 | CGGCACPCCACAPCCAAGACPGCGGPPACCPPGPAGGC | 62 |
| 5334-8_18 | >1000 | CGGCACPCCACAPCCAAGACPGCGGPPACCPPGPAG | 63 |
| 5334-8_19 | >1000 | CGGCACPCCACAPCCAAGACPGCGGPPACCPPGP | 64 |

The data from table 3 shows that SEQ ID Nos: 48, 49, 51, 51, 52, 53, 54, 55, 56 and 57 have K_{d} values from about 5.3 nM to about 25 nM indicating that certain 5′-end nucleotides and certain 3′-end nucleotides of the aptamer may be removed, and the aptamer retains binding capability to IgG. The data from Table 3 also indicates that the removal of more than 4 nucleotide from the 3-end of 5334-8_3 (see SEQ ID NOs: 58, 59, 60, 61, 62, 63 and 64), results in K_{d} values of greater than 1000 nM (or >1000 nM), which is considered to be a “no binding” (or NB) result for the dissociation constant assay.

To further understand the contribution of the 5′-end and 3′-end nucleotides of the 5334-8_3 (SEQ ID NO:48) aptamer to IgG binding, additional truncates were generated with both 5′ and 3′ end nucleotides removed. The K_{d} values for each aptamer is shown in Table 4. For table 4, “P” in each sequence represents a 2NapdU. The sequences in Table 4 are aligned to show how each truncated sequence overlaps with the parent 50-mer sequence (5334-8_3).

TABLE 4
5334-8 aptamer 5'-end and 3'-end truncation series

| Aptamer | Kd | Aptamer Nucleic Acid Sequence (5' to 3') | SEQ ID |
|---|---|---|---|
| ID | (nM) | (P is a 2NapdU) | NO: |
| 5334-8_3 | 12.2 | CGGCACPCCACAPCCAAGACPGCGGPPACCPPGPAGGCGAPPAPGGGCAG | 48 |
| 5334-8_21 | 6.77 | GACPGCGGPPACCPPGPAGGCGAPPAPGGGCAG | 65 |
| 5334-8_22 | 5.77 | ACPGCGGPPACCPPGPAGGCGAPPAPGGGCAG | 66 |
| 5334-8_23 | 4.04 | CPGCGGPPACCPPGPAGGCGAPPAPGGGCAG | 67 |
| 5334-8_24 | 5.51 | PGCGGPPACCPPGPAGGCGAPPAPGGGCAG | 68 |
| 5334-8_25 | 5.83 | GCGGPPACCPPGPAGGCGAPPAPGGGCAG | 69 |
| 5334-8_26 | >1000 | CGGPPACCPPGPAGGCGAPPAPGGGCAG | 70 |
| 5334-8_27 | 279 | GGPPACCPPGPAGGCGAPPAPGGGCAG | 71 |
| 5334-8_28 | 8.45 | GACPGCGGPPACCPPGPAGGCGAPPAPGGGCA | 72 |
| 5334-8_29 | 15.6 | AGACPGCGGPPACCPPGPAGGCGAPPAPGGGC | 73 |
| 5334-8_30 | 80.9 | AGACPGCGGPPACCPPGPAGGCGAPPAPGGG | 74 |
| 5334-8_31 | >1000 | AGACPGCGGPPACCPPGPAGGCGAPPAPGG | 75 |
| 5334-8_32 | >1000 | AGACPGCGGPPACCPPGPAGGCGAPPAP | 76 |
| 5334-8_33 | >1000 | AGACPGCGGPPACCPPGPAGGCGAPP | 77 |
| 5334-8_34 | 9.71 | GCGGPPACCPPGPAGGCGAPPAPGGGCA | 78 |

The data from Table 4 shows that SEQ ID Nos: 65, 66, 67, 68, 69, 71, 72, 73, 74 and 78 have K_{d} values from about 4 nM to about 279 nM indicating that 5′-end and 3′-end nucleotides of the aptamer may be removed, and the aptamer retains binding capability to IgG. The data from Table 4 also indicates that a 27-mer sequence (5334-8_27; SEQ ID NO: 71) is sufficient to bind IgG (K_{d} value of 279 nM). The following sequence is a “core” sequence sufficient to bind IgG (P is 2NapdU):

(SEQ ID NO: 71)
5'- GGPPACCPPGPAGGCGAPPAPGGGCAG -3'.

Thus, in general, an aptamer that comprises additional nucleotides on the 5′-end and/or the 3′-end of SEQ ID NO: 71 retains the ability to bind IgG as shown by the K_{d} values provided in Tables 3 and 4.

The data from Table 4 further indicates that a 28-mer sequence (5334-8_34; SEQ ID NO: 78) is sufficient to bind IgG (K_{d} value of 9.71 nM). The following sequence is a “core” sequence sufficient to bind IgG (P is 2NapdU):

(SEQ ID NO: 78)
5'- GCGGPPACCPPGPAGGCGAPPAPGGGCA -3'.

Additional “core” sequences sufficient to bind IgG include (P is 2NapdU):

(SEQ ID NO: 74)
5' - AGACPGCGGPPACCPPGPAGGCGAPPAPGGG - 3';
(SEQ ID NO: 69)
5' - GCGGPPACCPPGPAGGCGAPPAPGGGCAG - 3';

and

(SEQ ID NO: 73)

5' - AGACPGCGGPPACCPPGPAGGCGAPPAPGGGC - 3'.

Thus, an aptamer that comprises additional nucleotides on the 5′-end and/or the 3′-end of SEQ ID NOs:69, 73, 74 or 78 is expected to retain the ability to bind IgG as shown by the K_{d} values provided in Tables 3 and 4.

Table 5 shows the K_{d} values for the 14125-144_3 (50-mer; SEQ ID NO: 79) aptamer for IgG, and the 5′-end and 3′-end truncation analysis of the 50-mer. For Table 5, “P” in each sequence represents a NapdU. The sequences in Table 5 are aligned to show how each truncated sequence overlaps with the parent 50-mer sequence (14125-144_3).

TABLE 5
14125-144 aptamer truncation series (5' or 3' truncations)

| | Kd | Aptamer Nucleic Acid Sequence (5' to 3')? | SEQ ID |
|---|---|---|---|
| Aptamer ID | (nM) | (P is a NapdU) | NO: |
| 14125-144_3 | 16.3 | GCCTGCACGAPGGCGAACPCCCPGAAPGCPCPPGPCPPAAGPCCCCAACA | 79 |
| 14125-144_4 | 10.5 | CTGCACGAPGGCGAACPCCCPGAAPGCPCPPGPCPPAAGPCCCCAACA | 80 |
| 14125-144_5 | 8.87 | GCACGAPGGCGAACPCCCPGAAPGCPCPPGPCPPAAGPCCCCAACA | 81 |
| 14125-144_6 | 7.49 | ACGAPGGCGAACPCCCPGAAPGCPCPPGPCPPAAGPCCCCAACA | 82 |
| 14125-144_7 | 6.96 | GAPGGCGAACPCCCPGAAPGCPCPPGPCPPAAGPCCCCAACA | 83 |
| 14125-144_8 | 6.82 | PGGCGAACPCCCPGAAPGCPCPPGPCPPAAGPCCCCAACA | 84 |
| 14125-144_9 | >1000 | GCGAACPCCCPGAAPGCPCPPGPCPPAAGPCCCCAACA | 85 |
| 14125-144_10 | >1000 | GAACPCCCPGAAPGCPCPPGPCPPAAGPCCCCAACA | 86 |
| 14125-144_11 | >1000 | ACPCCCPGAAPGCPCPPGPCPPAAGPCCCCAACA | 87 |
| 14125-144_12 | 17.2 | GCCTGCACGAPGGCGAACPCCCPGAAPGCPCPPGPCPPAAGPCCCCAA | 88 |
| 14125-144_13 | 14.3 | GCCTGCACGAPGGCGAACPCCCPGAAPGCPCPPGPCPPAAGPCCCC | 89 |
| 14125-144_14 | 15.8 | GCCTGCACGAPGGCGAACPCCCPGAAPGCPCPPGPCPPAAGPCC | 90 |
| 14125-144_15 | 14.6 | GCCTGCACGAPGGCGAACPCCCPGAAPGCPCPPGPCPPAAGP | 91 |
| 14125-144_16 | 8.82 | GCCTGCACGAPGGCGAACPCCCPGAAPGCPCPPGPCPPAA | 92 |
| 14125-144_17 | 15.4 | GCCTGCACGAPGGCGAACPCCCPGAAPGCPCPPGPCPP | 93 |
| 14125-144_18 | >1000 | GCCTGCACGAPGGCGAACPCCCPGAAPGCPCPPGPC | 94 |
| 14125-144_19 | >1000 | GCCTGCACGAPGGCGAACPCCCPGAAPGCPCPPG | 95 |

The data from Table 5 shows that SEQ ID Nos: 79, 80, 81, 82, 83, 84, 88, 89, 90, 91, 92 and 93 have K_{d} values from about 6.8 nM to about 17 nM indicating that certain 5′-end nucleotides of the aptamer, and certain 3′-end nucleotides of the aptamer may be removed, and the aptamer retains binding capability to IgG. The data from Table 5 also indicates that the removal of more than 11 nucleotide from the 5′ end 14125-144-3 (see SEQ ID Nos: 85-87), and 13 nucleotides of 3′-end of 14125-144-3 (see SEQ ID NOs: 94 and 95), results in K_{d} values of greater than 1000 nM (or >1000 nM), which is considered to be a “no binding” (or NB) result for the dissociation constant assay.

To further understand the contribution of the 5′-end and 3′-end nucleotides of the 14125-144-3 (SEQ ID NO:79) aptamer to IgG binding, additional truncations were generated with both 5′ and 3′end nucleotides were removed. The K_{d} values for each aptamer is shown in Table 6. For table 6, “P” in each sequence represents a NapdU. The sequences in Table 6 are aligned to show how each truncated sequence overlaps with the parent 50-mer sequence (14125-144-3).

TABLE 6
14125-144 aptamer 5'-end and 3'-end truncation series

| | Kd | Aptamer Nucleic Acid Sequence (5' to 3')? | SEQ ID |
|---|---|---|---|
| Aptamer ID | (nM) | (P is a NapdU) | NO: |
| 14125-144_3 | 18.8 | GCCTGCACGAPGGCGAACPCCCPGAAPGCPCPPGPCPPAAGPCCCCAACA | 79 |
| 14125-144_20 | 6.67 | GAPGGCGAACPCCCPGAAPGCPCPPGPCPPAA | 96 |
| 14125-144_21 | 5.5 | APGGCGAACPCCCPGAAPGCPCPPGPCPPAA | 97 |
| 14125-144_22 | 3.55 | PGGCGAACPCCCPGAAPGCPCPPGPCPPAA | 98 |
| 14125-144_23 | >1000 | GGCGAACPCCCPGAAPGCPCPPGPCPPAA | 99 |
| 14125-144_24 | 9.89 | GAPGGCGAACPCCCPGAAPGCPCPPGPCPPA | 100 |
| 14125-144_25 | 7.55 | APGGCGAACPCCCPGAAPGCPCPPGPCPPA | 101 |
| 14125-144_26 | 5.03 | PGGCGAACPCCCPGAAPGCPCPPGPCPPA | 102 |
| 14125-144_27 | >1000 | GGCGAACPCCCPGAAPGCPCPPGPCPPA | 103 |
| 14125-144_28 | 17.2 | GAPGGCGAACPCCCPGAAPGCPCPPGPCPP | 104 |
| 14125-144_29 | 11.6 | APGGCGAACPCCCPGAAPGCPCPPGPCPP | 105 |
| 14125-144_30 | 8.18 | PGGCGAACPCCCPGAAPGCPCPPGPCPP | 106 |
| 14125-144_31 | >1000 | GGCGAACPCCCPGAAPGCPCPPGPCPP | 107 |
| 14125-144_32 | >1000 | GAPGGCGAACPCCCPGAAPGCPCPPGPCP | 108 |
| 14125-144_33 | >1000 | APGGCGAACPCCCPGAAPGCPCPPGPCP | 109 |
| 14125-144_34 | >1000 | PGGCGAACPCCCPGAAPGCPCPPGPCP | 110 |
| 14125-144_35 | >1000 | GGCGAACPCCCPGAAPGCPCPPGPCP | 111 |
| 14125-144_36 | >1000 | GAPGGCGAACPCCCPGAAPGCPCPPGPC | 112 |
| 14125-144_37 | >1000 | APGGCGAACPCCCPGAAPGCPCPPGPC | 113 |
| 14125-144_38 | >1000 | PGGCGAACPCCCPGAAPGCPCPPGPC | 114 |
| 14125-144_39 | >1000 | GGCGAACPCCCPGAAPGCPCPPGPC | 115 |

The data from Table 6 shows that SEQ ID Nos: 79, 96, 97, 98, 100, 101, 102, 104, 105 and 106 have K_{d} values from about 3.5 nM to about 18 nM indicating that certain 5′-end and 3′-end nucleotides of the aptamer may be removed, and the aptamer retains binding capability to IgG. The data from Table 6 also indicates that a 28-mer sequence (14125-144_30; SEQ ID NO: 106) is sufficient to bind IgG (K_{d} value of about 8 nM). The following sequence is a “core” sequence sufficient to bind IgG (P is NapdU):

(SEQ ID NO: 106)

5'- PGGCGAACPCCCPGAAPGCPCPPGPCPP -3.

Thus, an aptamer comprising additional nucleotides on the 5′-end and/or the 3′-end of SEQ ID NO:106 is expected to retain the ability to bind IgG as shown by the K_{d} values provided in Tables 5 and 6.

### Example 3

This example provides the binding affinities of the 5406-56_3; 5406-56_48; 5334-8_3; 5334-8_34; 14125-144_3 and 14125-144_30 aptamers for the four different human IgG subclasses (IgG₁, IgG₂, IgG₃ and IgG₄), each paired with a kappa light chain constant region, or as an Fc region, and the monkey, mouse, cow, goat, sheep and rabbit IgG proteins. The protocol used to measure the binding affinity (dissociation constant) of the aptamer for the protein is provided in Example 2.

Binding affinities for selected aptamers are shown in Tables 7 and 8 against total IgG, the subclasses of IgG, and other immunoglobulin classes (e.g., IgM, IgA and IgD). Table 7 shows the binding affinities for human IgG and other classes, while Table 8 shows the binding affinities for IgG and other classes from species other than human (monkey, mouse, cow, goat, sheep and rabbit).

TABLE 7
Binding affinities for select aptamers with a protein target.

| Antibody | 5334- | 5334- | 14125- | 14125- | 5406- | 5406- |
|---|---|---|---|---|---|---|
| Class (Human) | 8_3 | 8_34 | 144_3 | 144_30 | 56_3 | 56_48 |
| Total IgG | 1E−08 | 8E−09 | 2E−08 | 3E−08 | 6E−09 | 6E−09 |
| IgG₁-Kappa | 4E−08 | 2E−08 | 2E−08 | 4E−08 | 2E−09 | 2E−09 |
| IgG₁-Fc | NT | 5E−09 | 4E−08 | 3E−08 | 2E−09 | 3E−09 |
| β-FGFR fusion | 9E−09 | 2E−09 | 3E−09 | 4E−09 | 3E−09 | 4E−09 |
| IgG₂-Kappa | NB | NB | NB | NB | NB | NB |
| IgG₂-Fc | NT | 2E−09 | 8E−09 | 7E−09 | NB | NB |
| IgG₃-Kappa | 7E−09 | 4E−09 | 4E−09 | 8E−09 | 8E−08 | 1E−07 |
| IgG₃-Fc | NT | 1E−09 | 5E−09 | 2E−09 | 4E−07 | 7E−08 |
| IgG₄-Kappa | 1E−08 | 3E−09 | 5E−09 | 4E−09 | 2E−08 | 5E−08 |
| IgG₄-Fc | NT | 2E−09 | 2E−08 | 3E−08 | NB | 3E−08 |
| IgM | 1E−07 | 6E−10 | 4E−08 | 6E−09 | 1E−07 | 7E−08 |
| IgD | 6E−07 | 6E−08 | 6E−07 | 8E−08 | NB | 5E−07 |
| IgA | 2E−07 | 4E−08 | 3E−08 | 1E−07 | 2E−07 | 2E−07 |

NB is “no binding;
NT is “not tested”

TABLE 8

| Antibody | 5334- | 5334- | 14125- | 14125- | 5406- | 5406- |
|---|---|---|---|---|---|---|
| Class | 8_3 | 8_34 | 144_3 | 144_30 | 56_3 | 56_48 |
| Total IgG | 2E−08 | 6E−09 | 8E−08 | 6E−08 | NB | 1E−07 |
| (Monkey) | | | | | | |
| Total IgG | NT | 2E−07 | NT | NB | NT | NB |
| (Mouse) | | | | | | |
| IgG1 | NB | 3E07 | NB | NB | NB | NB |
| (Mouse) | | | | | | |
| Total IgG | NB | 5E−07 | NB | NB | NB | NB |
| (Cow) | | | | | | |
| IgM | NT | 4E−08 | NT | NB | NT | NB |
| (Cow) | | | | | | |
| Total IgG | NB | 6E−07 | NB | NB | NB | NB |
| (Goat) | | | | | | |
| Total IgG | NB | 5E−07 | NB | NB | NB | NB |
| (Sheep) | | | | | | |
| Total IgG | 2E−08 | 9E−09 | 4E−08 | 1E−07 | NB | NB |
| (Rabbit) | | | | | | |

NB is “no binding;
NT is “not tested”

### Example 4

This example provides the conditions and buffers for the elution of IgG proteins from IgG-aptamer affinity complexes.

In this Example, the method for detection of protein elution used a 96-well plate-based assay. A biotinylated anti-IgG-Fc aptamer (or SOMAmer) was captured on a 96 well streptavidin plate (SA Coated High Binding Capacity (HBC) clear 96 well plate with superblock blocking buffer, Pierce #15500) by adding 100 μL of a 1 μg/mL aptamer solution in HBS/0.01T or HBSE/0.01T to each well. (HBS=HEPES buffered saline, 125 mM NaCl, 25 mM HEPES, pH 7.3; HBSE=HBS+5 mM EDTA, pH 7; HBS/0.01T and HBSE/0.01T include 0.01% (v/v) Tween-20)). The plate was washed 3× by the addition of 300 μL wash buffer per well (HBS/0.01T or HBSE/0.01T), shaken to mix for 1 min at 450 rpm (Eppendorf Thermomixer), and emptied manually.

The plate was then incubated with IgG₁. 100 μL of a 5 μg/mL (in HBS/0.01T) protein stock was added per well, and the plate was shaken to mix for a minimum of 1 hour at 450 rpm. The plate was washed 2× by the addition of 300 μL wash buffer (HBS/0.01T or HBSE/0.01T) per well, shaken to mix for 1 min at 500 rpm, and the plate emptied manually.

Next, the aptamer-protein complex was exposed to an elution condition and washed. 100 lit of elution buffer (HBS/0.01T+additives) was added per well, shaken to mix for 2 min at 450 rpm, and the plate emptied manually. The protein elution was conducted twice and the order of addition was reversed on the second elution to equalize total elution time. The plate was washed 3× by the addition of 300 μL wash buffer (HBS/0.01T or HBSE/0.01T) per well, shaken to mix for 1 min at 450 rpm, and the plate emptied manually.

Each well was then exposed to horseradish peroxidase (HRP) protein G that binds to the Fc region of any IgG₁ remaining on the surface. 100 μL/well of a 1:1000 dilution of the reagent (HRP-rec-protein G, LifeTech #101223) in HBS/0.01T was used. The plate was shaken to mix for 45-60 min at 500 rpm. The plate was washed 5× with 300 μL wash buffer per well (HBS/0.01T or HBSE/0.01T), shaken to mix for 1 min at 450 rpm, and the plate emptied manually.

The presence of IgG₁ was revealed by the addition of 3,3′,5,5′-tetramethylbenzidine (TMB), which generated a blue color upon interaction with HRP. TMB substrate was added at 100 μL/well. (TMB Substrate Kit, Thermo #34021). Sulfuric acid (2 M H₂SO₄) was then added at 50 μL/well to quench this reaction and generate a yellow color which was detected by absorbance at 450 nm on a plate reader (SpectraMax). In this format, a weak or nonexistent signal is an indication that the elution conditions have been successful, though degradation of the protein could yield a false positive.

The elution buffer controls were HBS/T0.01% (negative control), and 1 M imidazole/2 M NaCl pH 9 in ½ strength HBS/T0.01% (positive control).

TABLE 9
UV 450 nm results from SA plate assay of
IgG₁ aptamers with elution solutions.

| | | Truncated Aptamer | | |
|---|---|---|---|---|
| | | Identifier | | |
| | | 5334- | 14125- | 5406- |
| Elution Condition | pH¹ | 8_34 | 144_30 | 56_48 |
| 0.1M Copper(II) chloride dihydrate | 4 (2) | 0.886 | 0.868 | 0.824 |
| (brown) | | | | |
| 2.0M Sodium chloride | 5 (2) | 0.846 | 1.220 | 0.299 |
| 0.5M Sodium fluoride | 7 | 0.771 | 1.257 | 0.286 |
| 1.0M Sodium iodide | 5 | 0.737 | 0.393 | 0.082 |
| 2.0M Sodium thiocyanate | 5 | 0.388 | 0.084 | 0.071 |
| 30% w/v 1,5-Diaminopentane | 3 | 0.716 | 0.388 | 0.141 |
| dihydrochloride | | | | |
| 30% w/v 1,6-Diaminohexane | 13 | 0.141 | 0.382 | 0.126 |
| 30% w/v 1,8-Diaminooctane | 12 | 0.114 | 0.325 | 0.149 |
| 0.1M Betaine hydrochloride | 2 | 0.346 | 0.402 | 0.445 |
| 0.1M Spermidine | 12 | 0.222 | 0.523 | 0.359 |
| 0.1M Spermine tetrahydrochloride | 3 | 1.206 | 1.122 | 0.206 |
| 0.1M β-Nicotinamide adenine | 3 | 0.601 | 0.537 | 0.258 |
| dinucleotide hydrate | | | | |
| 0.1M Adenosine-5′-triphosphate | 3 | 0.959 | 0.998 | 0.232 |
| disodium salt hydrate | | | | |
| 40% v/v Pentaerythritol ethoxylate | 5 | 0.161 | 1.335 | 0.108 |
| (3/4 EO/OH) | | | | |
| 2.0M NDSB-201 | 3 | 0.845 | 1.192 | 0.411 |
| 20% w/v Benzamidine hydrochloride | 5² | 0.084 | 0.086 | 0.084 |
| 5% w/v n-Dodecyl-N,N-dimethylamine- | 7 | 0.549 | 1.132 | 0.398 |
| N-oxide | | | | |
| 50% v/v Jeffamine M-600 pH 7.0 | 4 | 0.145 | 1.376 | 0.323 |
| 1M imidazole/2M NaCl pH9 | 10 | 0.100 | 0.103 | 0.100 |
| 1M imidazole/2M NaCl pH9 | 10 | 0.097 | 0.112 | 0.088 |
| 1M Benzoate | 8 | 1.367 | 1.341 | 0.114 |
| 5% v/v Ethyl acetate | 3 | 1.339 | 1.244 | 0.711 |
| 1M Benzoate | 8 | 1.357 | 1.375 | 0.184 |
| 40% v/v 1,1,1,3,3,3-Hexafluoro- | 4 (2) | 0.259 | 0.389 | 0.511 |
| 2-propanol | | | | |

¹pH checked by pH indicator paper 0-14
²white precipitate observed in well

TABLE 10
UV 450 nm results from SA plate assay of
IgG₁ aptamers with ionic elution solutions.

| | | | Truncated |
|---|---|---|---|
| | | | Aptamer |
| | | | Identifier |
| | Conc. | | 5406- |
| Elution Condition | (M) | pH¹ | 56_48 |
| Tetraethylammonium bromide | 1.2 | 5 | 0.086 |
| Benzyltriethylammonium chloride | 1.1 | 3 | 0.090 |
| 2-Hydroxyethylammonium formate | 2.3 | 6 | 0.263 |
| Ethylammonium nitrate | 2.3 | 4 | 0.071 |
| Cholin acetate | 1.5 | 6 | 0.120 |
| Choline dihydrogen phosphate | 1.2 | 5 | 0.408 |
| 1-Ethyl-3-methylimidazolium acetate | 1.5 | 7 | 0.074 |
| 1-Butyl-3-methylimidazolium chloride | 1.4 | 6 | 0.071 |
| 1-Ethyl-3-methylimidazolium chloride | 1.7 | 5 | 0.078 |
| 1-Hexyl-3-methylimidazolium chloride | 1.2 | 5 | 0.083 |
| 1-Butyl-3-methylimidazolium dicyanamide | 1.2 | 5 | 0.077 |
| 1,3-Dimethylimidazolium dimethyl phosphate | 1.1 | 4 | 0.481 |
| 1,3-Dimethylimidazolium methyl sulfate | 1.2 | 3 | 0.599 |
| 1-Butyl-3-methylimidazolium methyl sulfate | 1 | 3 | 0.343 |
| 1-n-Butyl-3-methylimidazolium n-octylsulfate | 0.7 | 7 | 0.081 |
| 1-Ethyl-3-methylimidazolium ethyl sulfate | 1.5 | 7 | 0.078 |
| 1-Ethyl-3-methylimidazolium tetrafluoroborate | 1.3 | 3 | 0.345 |
| 1-Butyl-2,3-dimethylimidazolium | 1 | 3 | 0.372 |
| tetrafluoroborate | | | |
| 1-Butyl-3-methylimidazolium tetrafluoroborate | 1.1 | 3 | 0.298 |
| 1-Butyl-3-methylimidazolium trifluoroacetate | 1 | 4 | 0.417 |
| 1-Ethyl-3-methylimidazolium | 1 | 3 | 0.344 |
| trifluoromethanesulfonate | | | |
| Tetrabutylphosphonium bromide | 0.7 | 2 | 0.182 |
| Triisobutylmethylphosphonium tosylate | 0.6 | 2 | 0.286 |
| 1-Butylpyridinium chloride | 1.5 | 4 | 0.072 |

¹pH checked by pH indicator paper 0-14;
2) Elution Conditions are from Hampton Research (Cat#: HR2-214; each ionic liquid is pre-formulated in deionized water)

Based on the elution performance with both benzamidine and the alkyl imidazolium derivatives (Table 11), further testing of these compounds was done. Structures for these compounds are shown below, including the resonance structures for the alkyl imidazolium derivatives. Elution of each aptamer truncate with combinations of benzamidine and alkyl imidazolium derivatives in HBS/0.01% Tween-20, pH 7, are shown below in Table 12. Elution buffer concentrations ranged from 300 mM down to 40 mM in either benzamidine, an imidazolium derivative, or both, diluted in 1.5× steps. The elution time was 10 minutes at 22° C. The positive control was 1 M imidazole 2 M NaCl, pH 9 and the negative control was HBS 0.01% Tween buffer.

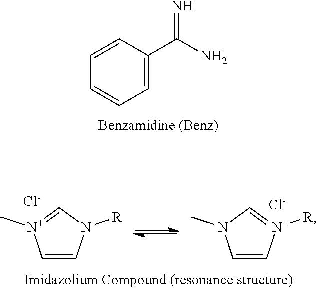

wherein R is selected from non-substituted alkyl, alkenyl, and benzyl. In some embodiments, R is selected from non-substituted C₁-C₁₂ alkyl, C₂-C₆ alkenyl, and benzyl. In some embodiments, R is selected from C₂-C₁₀ alkyl, C₂-C₄ alkenyl, and benzyl.

TABLE 11

| Alkyl Imidazolium derivative | R Group |
|---|---|
| 1-decyl-3-methylimidazolium chloride (DLM) | —(CH₂)₉CH₃ |
| 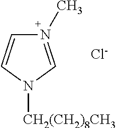 | |
| 1-methyl-3-octylimidazolium chloride (MOM) | —(CH₂)₇CH₃ |
| 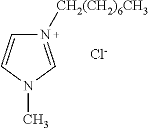 | |
| 1-hexyl-3-methylimidazolium chloride (HLM) | —(CH₂)₅CH₃ |
| 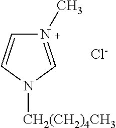 | |
| 1-benzyl-3-methylimidazolium chloride (BLM) | —CH₂—C₆H₅ |
| 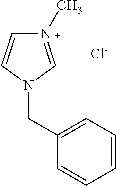 | |
| 1-butyl-3-methylimidazolium chloride (BUM) | —(CH₂)₃CH₃ |
| 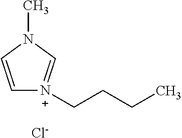 | |
| 1-allyl-3-methylimidazolium chloride (ALM) | —CH₂CH═CH₂ |
| 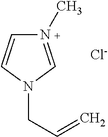 | |

TABLE 12

| | | | | | |
|---|---|---|---|---|---|
| | | | | BUM + | HLM + |
| Conc. | Benz | BUM | HLM | Benz | Benz |

5334-8_34

| | | | | | | |
|---|---|---|---|---|---|---|
| 300 | mM | 0.0766 | 0.8505 | 0.1295 | 0.0739 | 0.0692 |
| 200 | mM | 0.1098 | 0.8644 | 0.4179 | 0.0867 | 0.077 |
| 133 | mM | 0.1896 | 0.9304 | 0.6534 | 0.1358 | 0.1045 |
| 88 | mM | 0.4544 | 0.7489 | 0.8986 | 0.2751 | 0.2037 |
| 60 | mM | 0.7393 | 0.9122 | 1.0325 | 0.5855 | 0.4981 |
| 40 | mM | 0.857 | 0.9396 | 0.8658 | 0.8854 | 0.7881 |

| | | | | | |
|---|---|---|---|---|---|
| +Control | 0.087 | 0.0863 | 0.0906 | 0.0919 | 0.0917 |
| −Control | 1.0458 | 0.8837 | 0.9262 | 0.8751 | 0.9576 |

14125-144_30

| | | | | | | |
|---|---|---|---|---|---|---|
| 300 | mM | 0.2582 | 1.1822 | 0.1334 | 0.1794 | 0.093 |
| 200 | mM | 0.7054 | 1.1254 | 0.6251 | 0.6228 | 0.1455 |
| 133 | mM | 0.8971 | 1.226 | 0.9289 | 1.0156 | 0.5141 |
| 88 | mM | 1.1501 | 1.1347 | 1.1029 | 1.1989 | 1.0685 |
| 60 | mM | 1.1657 | 1.2872 | 1.2196 | 1.1743 | 1.0679 |
| 40 | mM | 1.2633 | 1.1176 | 1.279 | 1.348 | 1.0819 |

| | | | | | |
|---|---|---|---|---|---|
| +Control | 0.126 | 0.0774 | 0.0775 | 0.0764 | 0.0805 |
| −Control | 1.3638 | 1.2427 | 1.1852 | 1.3014 | 1.2703 |

5406-56_48

| 300 | mM | 0.111 | NT | 0.1079 | NT | 0.1045 |
|---|---|---|---|---|---|---|
| 200 | mM | 0.1149 | NT | 0.1193 | NT | 0.1037 |
| 133 | mM | 0.1245 | NT | 0.1264 | NT | 0.1237 |
| 88 | mM | 0.1802 | NT | 0.1945 | NT | 0.1129 |
| 60 | mM | 0.2729 | NT | 0.3266 | NT | 0.1201 |
| 40 | mM | 0.4741 | NT | 0.5129 | NT | 0.1855 |
| +Control | | 0.1307 | NT | 0.1381 | NT | 0.1202 |
| −Control | | 0.7947 | NT | 0.7778 | NT | 0.8333 |
| 1M | | 0.0887 | 0.0833 | 0.0765 | 0.0881 | 0.1385 |
| 333 | mM | 0.0944 | 0.0835 | 0.0855 | 0.0844 | 0.1475 |
| 111 | mM | 0.1268 | 0.248 | 0.1182 | 0.1011 | 0.112 |
| 37 | mM | 0.3991 | 0.501 | 0.3658 | 0.2959 | 0.207 |
| 12 | mM | 0.5766 | 0.6579 | 0.5655 | 0.5816 | 0.5374 |

| | | | | | |
|---|---|---|---|---|---|
| Blank | 0.6927 | 0.7175 | 0.7155 | 0.7305 | 0.7267 |
| +Control | 0.1022 | 0.0946 | 0.0971 | 0.0937 | 0.0924 |
| −Control | 0.7403 | 0.7003 | 0.7778 | 0.7519 | 0.7946 |

The data in Table 12 show that for each aptamer truncate, the buffers containing 1-hexyl-3-methylimidazolium chloride (Hexyl or HLM) and benzamidine (Benz) were the most effective eluants as a function of concentration, and the combination of benzamidine and an imidazolium compound was more effective than either component alone. Based on these data, additional imidazolium derivatives were tested, including 1-allyl-3-methylimidazolium chloride (ALM), 1-benzyl-3-methylimidazolium chloride (BLM), 1-methyl-3-octylimidazolium chloride (MOM), and 1-decyl-3-methylimidazolium chloride (DLM). Table 13 (FIGS. 4-19) shows the data for all four IgG aptamer truncates with the new imidazolium salts as well as combinations with benzamidine for elution in the streptavidin plate-based assay.

TABLE 13

| | | | | | | | | |
|---|---|---|---|---|---|---|---|---|
| | | | | | ALM + | BLM + | DLM + | MOM + |
| ALM | BLM | DLM | MOM | Benz | Benz | Benz | Benz | Benz |

5406-56_48

| | | | | | | | | | | |
|---|---|---|---|---|---|---|---|---|---|---|
| 300 | mM | 0.2374 | 0.175 | 0.1084 | 0.109 | 0.1252 | 0.219 | 0.1136 | 0.1313 | 0.1279 |
| 200 | mM | 0.2157 | 0.1065 | 0.1029 | 0.1297 | 0.1285 | 0.2775 | 0.1022 | 0.1222 | 0.1211 |
| 133 | mM | 0.2743 | 0.1105 | 0.1041 | 0.1156 | 0.1326 | 0.2657 | 0.1032 | 0.1085 | 0.1162 |
| 88 | mM | 0.4349 | 0.1351 | 0.0988 | 0.1171 | 0.1629 | 0.2164 | 0.1103 | 0.1188 | 0.1199 |
| 60 | mM | 0.6324 | 0.2304 | 0.0977 | 0.1201 | 0.2612 | 0.1846 | 0.1133 | 0.098 | 0.1289 |
| 40 | mM | 0.8258 | 0.4289 | 0.1016 | 0.1375 | 0.3904 | 0.2359 | 0.1656 | 0.1009 | 0.124 |

| | | | | | | | | | |
|---|---|---|---|---|---|---|---|---|---|
| +Control | 0.1291 | 0.1462 | 0.1263 | 0.1136 | 0.1109 | 0.1194 | 0.1245 | 0.1223 | 0.1208 |
| −Control | 1.1566 | 1.11 | 1.0256 | 0.8971 | 0.9007 | 0.977 | 0.8809 | 0.74 | 0.7374 |

5334-8_34

| | | | | | | | | | | |
|---|---|---|---|---|---|---|---|---|---|---|
| 300 | mM | 0.8829 | 0.0875 | 0.0841 | 0.1091 | 0.1014 | 0.1216 | 0.0929 | 0.0992 | 0.1035 |
| 200 | mM | 1.1074 | 0.1017 | 0.0776 | 0.13 | 0.1532 | 0.1536 | 0.092 | 0.0898 | 0.1127 |
| 133 | mM | 1.4317 | 0.2104 | 0.0816 | 0.1341 | 0.1969 | 0.2222 | 0.1036 | 0.0923 | 0.1204 |
| 88 | mM | 1.6099 | 0.7376 | 0.1476 | 0.1403 | 0.4317 | 0.3202 | 0.1572 | 0.0892 | 0.1144 |
| 60 | mM | 1.467 | 1.2901 | 0.0909 | 0.1404 | 1.0792 | 0.7037 | 0.3009 | 0.0933 | 0.1453 |
| 40 | mM | 1.4678 | 1.4564 | 0.1079 | 0.1695 | 1.2979 | 1.3551 | 0.833 | 0.108 | 0.1826 |

| | | | | | | | | | |
|---|---|---|---|---|---|---|---|---|---|
| +Control | 0.0949 | 0.1005 | 0.0999 | 0.1009 | 0.1107 | 0.097 | 0.1007 | 0.1121 | 0.0969 |
| −Control | 1.384 | 1.6905 | 1.6369 | 1.4305 | 1.3555 | 1.4688 | 1.43 | 1.3493 | 1.2899 |

14125-144_30

| | | | | | | | | | | |
|---|---|---|---|---|---|---|---|---|---|---|
| 300 | mM | 1.0627 | 0.0748 | 0.3482 | 0.0704 | 0.1833 | 0.131 | 0.0681 | 0.0773 | 0.0766 |
| 200 | mM | 1.1418 | 0.1318 | 0.064 | 0.0756 | 0.6855 | 0.377 | 0.0851 | 0.0701 | 0.0693 |
| 133 | mM | 1.0548 | 0.5512 | 0.0584 | 0.0746 | 0.9892 | 1.0555 | 0.1659 | 0.0811 | 0.0716 |
| 88 | mM | 1.1112 | 0.964 | 0.0595 | 0.0758 | 1.0796 | 1.0687 | 0.6352 | 0.062 | 0.0745 |
| 60 | mM | 1.0974 | 1.017 | 0.0568 | 0.0842 | 1.0295 | 1.1792 | 0.9016 | 0.0705 | 0.0783 |
| 40 | mM | 1.0066 | 1.0184 | 0.0606 | 0.125 | 0.9795 | 1.1861 | 0.8187 | 0.0623 | 0.1038 |

| | | | | | | | | | |
|---|---|---|---|---|---|---|---|---|---|
| +Control | 0.0615 | 0.0595 | 0.0595 | 0.0678 | 0.0662 | 0.0698 | 0.0628 | 0.0594 | 0.0601 |
| −Control | 0.7986 | 0.9736 | 0.9084 | 0.8439 | 0.8478 | 0.9598 | 0.9716 | 0.8828 | 0.9709 |

The data in Table 13 show similar trends to those observed in Table 12. Both ALM and BLM in combination with benzamidine were more effective than the compounds alone. DLM and MOM worked well at 40 mM both individually and in combination with benzamidine. Consequently, these compounds were tested at a lower concentration range (40-1.25 mM at 2× dilution steps). Table 14 shows these results, where DLM eluted at 10 mM and MOM eluted at 40 mM.

TABLE 14

| | | 5406-56_48 | | 5334-8_34 | | 14125-144_30 | |
|---|---|---|---|---|---|---|---|
| | | DLM | MOM | DLM | MOM | DLM | MOM |
| 40 | mM | 0.076 | 0.1237 | 0.0868 | 0.2373 | 0.0729 | 0.1532 |
| 20 | mM | 0.1113 | 0.2956 | 0.1076 | 0.9112 | 0.0781 | 1.0504 |
| 10 | mM | 0.2153 | 0.3378 | 0.2325 | 0.8098 | 0.0924 | 1.0667 |
| 5 | mM | 0.4079 | 0.356 | 0.8788 | 0.9297 | 1.1789 | 1.0817 |
| 2.5 | mM | 0.41 | 0.3855 | 1.0374 | 0.8821 | 0.9967 | 0.9977 |
| 1.25 | mM | 0.4345 | 0.4377 | 0.9774 | 0.9235 | 1.1421 | 1.0003 |

| | | | | | | |
|---|---|---|---|---|---|---|
| +Control | 0.0898 | 0.0907 | 0.0882 | 0.0804 | 0.0665 | 0.0701 |
| −Control | 0.4719 | 0.4434 | 0.9327 | 0.9643 | 1.0257 | 1.092 |

### Example 5

This example measures the binding activity of the IgG proteins that were eluted from aptamers with the alkyl imidazolium derivatives and benzamidine formulations. For these studies, the aptamer identified as aptamer-2744 was used. Aptamer-2744-57_37 is a 48-mer sequence having nineteen 5-position modified pyrimidines (e.g., BndU), and a binding affinity for human total IgG of 7.5 nM.

An agarose bead format assay was used to capture the IgG protein, and then eluted for functional activity testing.

Biotin labeled C-5 modified aptamers were immobilized on streptavidin beads (50 pmol aptamer (heat/cool). The beads were incubated for 20 minutes, shaken at 850 rpm at 25° C., washed 2× with CAPS and 2× with SB17/0.05% Tween-20. 50 pmol of IgG₁ full length protein was added. with 20 μM oligonucleotide having the following sequence (A-C-BndU-BndU)₇A-C. The beads, SOMAmer, and protein were incubated for 2 hours and shaken at 850 rpm at 28° C. 10 mM dextran sulfate was added for 5 minutes, shaken at 800 rpm at 25° C., and washed 6× with SB17/0.05% Tween-20. Elution buffer was added, the beads were incubated for 12 minutes, shaken 800 rpm at 25° C., and spun at 1000 rpm for 1 minute.

Immediately after the pull down, the eluted proteins were buffer exchanged into SB17/0.05% Tween 20 buffer using a Zeba 96-well spin plate (7K MWCO, 550 and then a Zorbax affinity assay was done using the eluted protein. Approximately 2 pmols of eluted protein was labeled with 0.1 mM NHS-Alexa 647 and run on a gel. Eluted protein concentration was roughly estimated from the relative band intensity, compared to a standard curve, because it was previously determined that no loss occurred during the buffer exchange. Data are summarized in Table 15. For IgG₁ full length, it appeared that the protein activity was not significantly affected by the elution conditions.

TABLE 15
IgG Protein Activity Post Elution from Aptamer-2744

| Target | Elution Conditions | K_{d} (nM) |
|---|---|---|
| IgG | 20 mM DLM, HBS, 0.01% Tween-20, pH 7 | 4 |
| IgG | 80 mM MOM, HBS, 0.01% Tween-20, | 4 |
| | pH 7 | |
| IgG | 150 mM BLM, 150 mM benzamidine, | 3 |
| | HBS, 0.01% Tween-20, pH 7 | |
| IgG | 300 mM HLM, 300 mM benzamidine, | 4 |
| | HBS, 0.01% Tween-20, pH 7 | |
| IgG | 500 mM ALM, 500 mM benzamidine, | 5 |
| | HBS, 0.01% Tween-20, pH 7 | |
| IgG | No Treatment | 6 |

What is claimed is:

1. An aptamer comprising a nucleobase sequence selected from the group consisting of SEQ ID NOs: 1-6, 10-16, 18-34, 36-47, 48-57, 65-69, 71-74, 78, 79-84, 88-93, 96-98, 100-102 and 104-106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

2. An aptamer comprising the nucleobase sequence selected from SEQ ID NOs: 45, 46 and 47, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

3. An aptamer comprising the nucleobase sequence selected from SEQ ID NOs: 69, 74 and 78, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

4. An aptamer comprising the nucleobase sequence of SEQ ID NO: 106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

5. The aptamer of claim 1, 2, 3, or 4, wherein the aptamer binds IgG with an affinity greater than 50 nM, or greater than 100 nM, or greater than 150 nM, or greater than 200 nM, or greater than 250 nM, or greater than 300 nM.

6. The aptamer of claim 1, 2, 3, or 4, wherein the aptamer binds IgG with an affinity less than 8 nM, or less than 7 nM, or less than 6 nM, or less than 5 nM, or less than 4 nM, or less than 3 nM, or less than 2 nM, or less than 1 nM.

7. The aptamer of claim 1, 2, 3, or 4, wherein the C-5 modified pyrimidine containing nucleoside is selected from the group consisting of 5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N—R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

8. The aptamer of claim 1, 2, 3, or 4, wherein the C-5 modified pyrimidine containing nucleoside is selected from a 5-(N-naphthylmethylcarboxyamide)-2′-deoxyuridine (NapdU) and a 5-(N-2-naphthylmethylcarboxyamide)-2′-deoxyuridine (2NapdU).

9. The aptamer of claim 1, 2, 3, or 4, wherein the 5′-end of the nucleotide sequence further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

10. The aptamer of claim 1, 2, 3, or 4, wherein the 3′-end of the nucleotide sequence further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

11. The aptamer of claim 1, 2, 3, or 4, wherein the 5′-end and the 3′-end, independently, of the nucleotide sequence further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

12. The aptamer of claim 2 or 4, wherein the C-5 modified pyrimidine containing nucleoside is a 5-(N-naphthylmethylcarboxyamide)-2′-deoxyuridine (NapdU).

13. The aptamer of claim 3, wherein the C-5 modified pyrimidine containing nucleoside is a 5-(N-2-naphthylmethylcarboxyamide)-2′-deoxyuridine (2NapdU).

14. The aptamer of any one of the preceding claims, wherein aptamer binds an IgG protein selected from IgG₁, IgG₂, IgG₃ and IgG₄.

15. The aptamer of any one of the preceding claims, wherein aptamer binds an IgG protein selected from human IgG protein, monkey IgG protein, mouse IgG protein, cow IgG protein, goat IgG protein, sheep IgG protein and rabbit IgG protein.

16. The aptamer of any one of the preceding claims, wherein the aptamer is at least from 27 to 100 nucleotides in length (or from 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length).

17. The aptamer of any one of the preceding claims, wherein at least one nucleotide of the nucleotide sequence comprises a 2′-O-methyl modification.

18. The aptamer of any one of the preceding claims, wherein at least one internucleoside linkage of the nucleotide sequence is a phosphorothioate.

19. A composition comprising an IgG protein and an aptamer comprising the nucleobase sequence selected from the group consisting of SEQ ID NOs: 1-6, 10-16, 18-34, 36-47, 48-57, 65-69, 71-74, 78, 79-84, 88-93, 96-98, 100-102 and 104-106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

20. A composition comprising an IgG protein and an aptamer comprising the nucleobase sequence selected from the group consisting of SEQ ID NOs: 45, 46 and 47, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

21. A composition comprising an IgG protein and an aptamer comprising the nucleobase sequence selected from the group consisting of SEQ ID NOs: 69, 74 and 78, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

22. A composition comprising an IgG protein and an aptamer comprising the nucleobase sequence selected from the group consisting of SEQ ID NO: 106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

23. The composition of claim 19, 20, 21, or 22, wherein the C-5 modified pyrimidine containing nucleoside is selected from the group consisting of 5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N—R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

24. The composition of claim 19, 20, 21, or 22, wherein the C-5 modified pyrimidine containing nucleoside is selected from a 5-(N-naphthylmethylcarboxyamide)-2′-deoxyuridine (NapdU) and a 5-(N-2-naphthylmethylcarboxyamide)-2′-deoxyuridine (2NapdU).

25. The composition of claim 19, 20, 21, or 22, wherein the 5′-end of the nucleotide sequence further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

26. The composition of claim 19, 20, 21, or 22, wherein the 3′-end of the nucleotide sequence further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

27. The composition of claim 19, 20, 21, or 22, wherein the 5′-end and the 3′-end, independently, of the nucleotide sequence further comprises from 1 to 50 nucleotides (or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

28. The composition of claim 20 or 22, wherein the C-5 modified pyrimidine containing nucleoside is a 5-(N-naphthylmethylcarboxyamide)-2′-deoxyuridine (NapdU).

29. The composition of claim 21, wherein the C-5 modified pyrimidine containing nucleoside is a 5-(N-2-naphthylmethylcarboxyamide)-2′-deoxyuridine (2NapdU).

30. An composition of any one of claims 19 to 29, wherein the aptamer is wherein the aptamer is at least from 27 to 100 nucleotides in length (or from 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length).

31. An aptamer of any one of claims 1-18, wherein one or more Pin the nucleobase sequence of the aptamer are a uracil.

32. An aptamer of any one of claims 1-18, wherein each P in the nucleobase sequence of the aptamer is a C-5 modified pyrimidine comprising a napthyl substituent covalently linked via a linker to the C-5 position of the pyrimidine base.

33. The aptamer of claim 32, wherein the linker is selected from the group consisting of an amide linker, a carbonyl linker, a propynyl linker, an alkyne linker, an ester linker, a urea linker, a carbamate linker, a guanidine linker, an amidine linker, a sulfoxide linker, and a sulfone linker and a combination thereof.

34. A composition of any one of claims 19-30, wherein one or more P positions of the aptamer are a uracil.

35. A composition of any one of claims 19-30, wherein each Pin the nucleobase sequence of the aptamer is a C-5 modified pyrimidine comprising a napthyl substituent covalently linked via a linker to the C-5 position of the pyrimidine base.

36. The composition of claim 35, wherein the linker is selected from the group consisting of an amide linker, a carbonyl linker, a propynyl linker, an alkyne linker, an ester linker, a urea linker, a carbamate linker, a guanidine linker, an amidine linker, a sulfoxide linker, and a sulfone linker and a combination thereof.

37. A method of purifying an IgG protein from a sample comprising the steps of:
a) incubating the sample with the aptamer of any one of claims 1 to 18 to produce an IgG protein-aptamer complex; and
b) eluting the IgG protein from the complex.

38. The method of claim 37 wherein the elution is performed in the presence of benzamidine, an alkyl imidazolium derivative, or a combination thereof.

39. The method of claim 38 wherein the alkyl imidazolium derivative is selected from the group consisting of 1-decyl-3-methylimidazolium chloride, 1-methyl-3-octylimidazolium chloride, 1-hexyl-3-methylimidazolium chloride, 1-benzyl-3-methylimidazolium chloride, 1-butyl-3-methylimidazolium chloride, and 1-allyl-3-methylimidazolium chloride.

40. A method of purifying an IgG protein from a sample comprising the steps of:
a) incubating the sample with an aptamer having affinity for the IgG protein to produce an IgG protein-aptamer complex; and
b) eluting the IgG protein from the complex in the presence of benzamidine, an alkyl imidazolium derivative, or a combination thereof.

41. The method of claim 40, wherein the alkyl imidazolium derivative has the resonance structure: 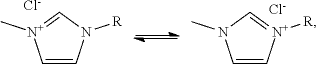
wherein R is selected from the group consisting of non-substituted alkyl, alkenyl, and benzyl.

42. The method of claim 41, wherein R is selected from the group consisting of non-substituted C₁-C₁₂ alkyl, C₂-C₆ alkenyl, and benzyl.

43. The method of claim 41, wherein R is selected from the group consisting of C₂-C₁₀ alkyl, C₂-C₄ alkenyl, and benzyl.

44. The method of claim 40, wherein the alkyl imidazolium derivative is selected from the group consisting of 1-decyl-3-methylimidazolium chloride, 1-methyl-3-octylimidazolium chloride, 1-hexyl-3-methylimidazolium chloride, 1-benzyl-3-methylimidazolium chloride, 1-butyl-3-methylimidazolium chloride, and 1-allyl-3-methylimidazolium chloride.

45. The method of any one of claims 40-44, wherein the aptamer comprises a nucleobase sequence selected from the group consisting of SEQ ID NOs: 1-6, 10-16, 18-34, 36-47, 48-57, 65-69, 71-74, 78, 79-84, 88-93, 96-98, 100-102 and 104-106, or a nucleobase sequence having at least 80%, at least 85%, at least 90%, at least 95%, or at least 99% identity thereto, wherein the P in the nucleobase sequence of the aptamer is, independently, for each occurrence, selected from the group consisting of a pyrimidine and a C-5 modified pyrimidine.

46. The method of claim 45, wherein one or more P in the nucleobase sequence of the aptamer are a uracil.

47. The method of claim 45, wherein each P in the nucleobase sequence of the aptamer is a C-5 modified pyrimidine comprising a napthyl substituent covalently linked via a linker to the C-5 position of the pyrimidine base.

48. The method of claim 47, wherein the linker is selected from the group consisting of an amide linker, a carbonyl linker, a propynyl linker, an alkyne linker, an ester linker, a urea linker, a carbamate linker, a guanidine linker, an amidine linker, a sulfoxide linker, and a sulfone linker and a combination thereof.

49. A method of purifying a protein from a sample comprising the steps of:
a) incubating the sample with an aptamer capable of binding the protein to produce a protein-aptamer complex; and
b) eluting the protein from the complex in the presence of benzamidine, an alkyl imidazolium derivative, or a combination thereof.

50. The method of claim 49, wherein the alkyl imidazolium derivative has the resonance structure: 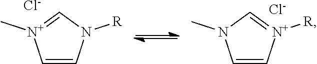
wherein R is selected from the group consisting of non-substituted alkyl, alkenyl, and benzyl.

51. The method of claim 50, wherein R is selected from the group consisting of non-substituted C₁-C₁₂ alkyl, C₂-C₆ alkenyl, and benzyl.

52. The method of claim 50, wherein R is selected from the group consisting of C₂-C₁₀ alkyl, C₂-C₄ alkenyl, and benzyl.

53. The method of claim 49, wherein the alkyl imidazolium derivative is selected from the group consisting of 1-decyl-3-methylimidazolium chloride, 1-methyl-3-octylimidazolium chloride, 1-hexyl-3-methylimidazolium chloride, 1-benzyl-3-methylimidazolium chloride, 1-butyl-3-methylimidazolium chloride, and 1-allyl-3-methylimidazolium chloride.

54. The method of any one of claims 49 to 53, wherein the aptamer comprises at least one C-5 modified pyrimidine.

55. The method of claim 54, wherein the C-5 modified pyrimidine containing nucleoside is selected from the group consisting of
5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N—R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

56. The method of any one of claims 49 to 55, wherein the aptamer comprises a detectable label.

57. The method of any one of claims 49 to 56, wherein the aptamer is bound to a solid support.

58. The method of any one of claims 49 to 56, wherein the aptamer comprises a member of a binding pair capable of being captured on a solid support.

59. The method of claim 58, wherein the aptamer is biotinylated.

60. The method of claim 58, wherein the solid support comprises streptavidin.

61. The method of any one of claims 49 to 60, wherein the protein is selected from an immunoglobulin protein, a domain of an immunoglobulin protein, an Fc region of an antibody, a Fab region of an antibody, an IgA, an IgD, and IgE, and IgG and an IgM.


SEQUENCE LISTING (115 sequences)

SEQ ID NO: 1 - DNA, 50 bp, Artificial Sequence
  Synthetic 5406-56_3
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
  misc_feature (38)..(38): n is NapdU
  misc_feature (42)..(42): n is NapdU
cctccanacggnacgannnncagnngganccagnaccnggcnggagagaa

SEQ ID NO: 2 - DNA, 48 bp, Artificial Sequence
  Synthetic 5406-56_4
  misc_feature (5)..(5): n is NapdU
  misc_feature (10)..(10): n is NapdU
  misc_feature (15)..(18): n is NapdU
  misc_feature (22)..(23): n is NapdU
  misc_feature (27)..(27): n is NapdU
  misc_feature (32)..(32): n is NapdU
  misc_feature (36)..(36): n is NapdU
  misc_feature (40)..(40): n is NapdU
tccanacggnacgannnncagnngganccagnaccnggcnggagagaa

SEQ ID NO: 3 - DNA, 46 bp, Artificial Sequence
  Synthetic 5406-56_5
  misc_feature (3)..(3): n is NapdU
  misc_feature (8)..(8): n is NapdU
  misc_feature (13)..(16): n is NapdU
  misc_feature (20)..(21): n is NapdU
  misc_feature (25)..(25): n is NapdU
  misc_feature (30)..(30): n is NapdU
  misc_feature (34)..(34): n is NapdU
  misc_feature (38)..(38): n is NapdU
canacggnacgannnncagnngganccagnaccnggcnggagagaa

SEQ ID NO: 4 - DNA, 44 bp, Artificial Sequence
  Synthetic 5406-56_6
  misc_feature (1)..(1): n is NapdU
  misc_feature (6)..(6): n is NapdU
  misc_feature (11)..(14): n is NapdU
  misc_feature (18)..(19): n is NapdU
  misc_feature (23)..(23): n is NapdU
  misc_feature (28)..(28): n is NapdU
  misc_feature (32)..(32): n is NapdU
  misc_feature (36)..(36): n is NapdU
nacggnacgannnncagnngganccagnaccnggcnggagagaa

SEQ ID NO: 5 - DNA, 42 bp, Artificial Sequence
  Synthetic 5406-56_7
  misc_feature (4)..(4): n is NapdU
  misc_feature (9)..(12): n is NapdU
  misc_feature (16)..(17): n is NapdU
  misc_feature (21)..(21): n is NapdU
  misc_feature (26)..(26): n is NapdU
  misc_feature (30)..(30): n is NapdU
  misc_feature (34)..(34): n is NapdU
cggnacgannnncagnngganccagnaccnggcnggagagaa

SEQ ID NO: 6 - DNA, 40 bp, Artificial Sequence
  Synthetic 5406-56_8
  misc_feature (2)..(2): n is NapdU
  misc_feature (7)..(10): n is NapdU
  misc_feature (14)..(15): n is NapdU
  misc_feature (19)..(19): n is NapdU
  misc_feature (24)..(24): n is NapdU
  misc_feature (28)..(28): n is NapdU
  misc_feature (32)..(32): n is NapdU
gnacgannnncagnngganccagnaccnggcnggagagaa

SEQ ID NO: 7 - DNA, 38 bp, Artificial Sequence
  Synthetic 5406-56_9
  misc_feature (5)..(8): n is NapdU
  misc_feature (12)..(13): n is NapdU
  misc_feature (17)..(17): n is NapdU
  misc_feature (22)..(22): n is NapdU
  misc_feature (26)..(26): n is NapdU
  misc_feature (30)..(30): n is NapdU
acgannnncagnngganccagnaccnggcnggagagaa

SEQ ID NO: 8 - DNA, 36 bp, Artificial Sequence
  Synthetic 5406-56_10
  misc_feature (3)..(6): n is NapdU
  misc_feature (10)..(11): n is NapdU
  misc_feature (15)..(15): n is NapdU
  misc_feature (20)..(20): n is NapdU
  misc_feature (24)..(24): n is NapdU
  misc_feature (28)..(28): n is NapdU
gannnncagnngganccagnaccnggcnggagagaa

SEQ ID NO: 9 - DNA, 34 bp, Artificial Sequence
  Synthetic 5406-56_11
  misc_feature (1)..(4): n is NapdU
  misc_feature (8)..(9): n is NapdU
  misc_feature (13)..(13): n is NapdU
  misc_feature (18)..(18): n is NapdU
  misc_feature (22)..(22): n is NapdU
  misc_feature (26)..(26): n is NapdU
nnnncagnngganccagnaccnggcnggagagaa

SEQ ID NO: 10 - DNA, 48 bp, Artificial Sequence
  Synthetic 5406-56_12
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
  misc_feature (38)..(38): n is NapdU
  misc_feature (42)..(42): n is NapdU
cctccanacggnacgannnncagnngganccagnaccnggcnggagag

SEQ ID NO: 11 - DNA, 46 bp, Artificial Sequence
  Synthetic 5406-56_13
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
  misc_feature (38)..(38): n is NapdU
  misc_feature (42)..(42): n is NapdU
cctccanacggnacgannnncagnngganccagnaccnggcnggag

SEQ ID NO: 12 - DNA, 44 bp, Artificial Sequence
  Synthetic 5406-56_14
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
  misc_feature (38)..(38): n is NapdU
  misc_feature (42)..(42): n is NapdU
cctccanacggnacgannnncagnngganccagnaccnggcngg

SEQ ID NO: 13 - DNA, 42 bp, Artificial Sequence
  Synthetic 5406-56_15
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
  misc_feature (38)..(38): n is NapdU
  misc_feature (42)..(42): n is NapdU
cctccanacggnacgannnncagnngganccagnaccnggcn

SEQ ID NO: 14 - DNA, 40 bp, Artificial Sequence
  Synthetic 5406-56_16
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
  misc_feature (38)..(38): n is NapdU
cctccanacggnacgannnncagnngganccagnaccngg

SEQ ID NO: 15 - DNA, 38 bp, Artificial Sequence
  Synthetic 5406-56_17
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
  misc_feature (38)..(38): n is NapdU
cctccanacggnacgannnncagnngganccagnaccn

SEQ ID NO: 16 - DNA, 36 bp, Artificial Sequence
  Synthetic 5406-56_18
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
cctccanacggnacgannnncagnngganccagnac

SEQ ID NO: 17 - DNA, 34 bp, Artificial Sequence
  Synthetic 5406-56_19
  misc_feature (7)..(7): n is NapdU
  misc_feature (12)..(12): n is NapdU
  misc_feature (17)..(20): n is NapdU
  misc_feature (24)..(25): n is NapdU
  misc_feature (29)..(29): n is NapdU
  misc_feature (34)..(34): n is NapdU
cctccanacggnacgannnncagnngganccagn

SEQ ID NO: 18 - DNA, 34 bp, Artificial Sequence
  Synthetic 5406-56_21
  misc_feature (1)..(1): n is NapdU
  misc_feature (6)..(6): n is NapdU
  misc_feature (11)..(14): n is NapdU
  misc_feature (18)..(19): n is NapdU
  misc_feature (23)..(23): n is NapdU
  misc_feature (28)..(28): n is NapdU
  misc_feature (32)..(32): n is NapdU
nacggnacgannnncagnngganccagnaccngg

SEQ ID NO: 19 - DNA, 33 bp, Artificial Sequence
  Synthetic 5406-56_22
  misc_feature (5)..(5): n is NapdU
  misc_feature (10)..(13): n is NapdU
  misc_feature (17)..(18): n is NapdU
  misc_feature (22)..(22): n is NapdU
  misc_feature (27)..(27): n is NapdU
  misc_feature (31)..(31): n is NapdU
acggnacgannnncagnngganccagnaccngg

SEQ ID NO: 20 - DNA, 32 bp, Artificial Sequence
  Synthetic 5406-56_23
  misc_feature (4)..(4): n is NapdU
  misc_feature (9)..(12): n is NapdU
  misc_feature (16)..(17): n is NapdU
  misc_feature (21)..(21): n is NapdU
  misc_feature (26)..(26): n is NapdU
  misc_feature (30)..(30): n is NapdU
cggnacgannnncagnngganccagnaccngg

SEQ ID NO: 21 - DNA, 31 bp, Artificial Sequence
  Synthetic 5406-56_24
  misc_feature (3)..(3): n is NapdU
  misc_feature (8)..(11): n is NapdU
  misc_feature (15)..(16): n is NapdU
  misc_feature (20)..(20): n is NapdU
  misc_feature (25)..(25): n is NapdU
  misc_feature (29)..(29): n is NapdU
ggnacgannnncagnngganccagnaccngg

SEQ ID NO: 22 - DNA, 30 bp, Artificial Sequence
  Synthetic 5406-56_25
  misc_feature (2)..(2): n is NapdU
  misc_feature (7)..(10): n is NapdU
  misc_feature (14)..(15): n is NapdU
  misc_feature (19)..(19): n is NapdU
  misc_feature (24)..(24): n is NapdU
  misc_feature (28)..(28): n is NapdU
gnacgannnncagnngganccagnaccngg

SEQ ID NO: 23 - DNA, 29 bp, Artificial Sequence
  Synthetic 5406-56_26
  misc_feature (1)..(1): n is NapdU
  misc_feature (6)..(9): n is NapdU
  misc_feature (13)..(14): n is NapdU
  misc_feature (18)..(18): n is NapdU
  misc_feature (23)..(23): n is NapdU
  misc_feature (27)..(27): n is NapdU
nacgannnncagnngganccagnaccngg

SEQ ID NO: 24 - DNA, 33 bp, Artificial Sequence
  Synthetic 5406-56_27
  misc_feature (1)..(1): n is NapdU
  misc_feature (6)..(6): n is NapdU
  misc_feature (11)..(14): n is NapdU
  misc_feature (18)..(19): n is NapdU
  misc_feature (23)..(23): n is NapdU
  misc_feature (28)..(28): n is NapdU
  misc_feature (32)..(32): n is NapdU
nacggnacgannnncagnngganccagnaccng

SEQ ID NO: 25 - DNA, 32 bp, Artificial Sequence
  Synthetic 5406-56_28
  misc_feature (5)..(5): n is NapdU
  misc_feature (10)..(13): n is NapdU
  misc_feature (17)..(18): n is NapdU
  misc_feature (22)..(22): n is NapdU
  misc_feature (27)..(27): n is NapdU
  misc_feature (31)..(31): n is NapdU
acggnacgannnncagnngganccagnaccng

SEQ ID NO: 26 - DNA, 31 bp, Artificial Sequence
  Synthetic 5406-56_29
  misc_feature (4)..(4): n is NapdU
  misc_feature (9)..(12): n is NapdU
  misc_feature (16)..(17): n is NapdU
  misc_feature (21)..(21): n is NapdU
  misc_feature (26)..(26): n is NapdU
  misc_feature (30)..(30): n is NapdU
cggnacgannnncagnngganccagnaccng

SEQ ID NO: 27 - DNA, 30 bp, Artificial Sequence
  Synthetic 5406-56_30
  misc_feature (3)..(3): n is NapdU
  misc_feature (8)..(11): n is NapdU
  misc_feature (15)..(16): n is NapdU
  misc_feature (20)..(20): n is NapdU
  misc_feature (25)..(25): n is NapdU
  misc_feature (29)..(29): n is NapdU
ggnacgannnncagnngganccagnaccng

SEQ ID NO: 28 - DNA, 29 bp, Artificial Sequence
  Synthetic 5406-56_31
  misc_feature (2)..(2): n is NapdU
  misc_feature (7)..(10): n is NapdU
  misc_feature (14)..(15): n is NapdU
  misc_feature (19)..(19): n is NapdU
  misc_feature (24)..(24): n is NapdU
  misc_feature (28)..(28): n is NapdU
gnacgannnncagnngganccagnaccng

SEQ ID NO: 29 - DNA, 28 bp, Artificial Sequence
  Synthetic 5406-56_32
  misc_feature (1)..(1): n is NapdU
  misc_feature (6)..(9): n is NapdU
  misc_feature (13)..(14): n is NapdU
  misc_feature (18)..(18): n is NapdU
  misc_feature (23)..(23): n is NapdU
  misc_feature (27)..(27): n is NapdU
nacgannnncagnngganccagnaccng

SEQ ID NO: 30 - DNA, 32 bp, Artificial Sequence
  Synthetic 5406-56_33
  misc_feature (1)..(1): n is NapdU
  misc_feature (6)..(6): n is NapdU
  misc_feature (11)..(14): n is NapdU
  misc_feature (18)..(19): n is NapdU
  misc_feature (23)..(23): n is NapdU
  misc_feature (28)..(28): n is NapdU
  misc_feature (32)..(32): n is NapdU
nacggnacgannnncagnngganccagnaccn

SEQ ID NO: 31 - DNA, 31 bp, Artificial Sequence
  Synthetic 5406-56_34
  misc_feature (5)..(5): n is NapdU
  misc_feature (10)..(13): n is NapdU
  misc_feature (17)..(18): n is NapdU
  misc_feature (22)..(22): n is NapdU
  misc_feature (27)..(27): n is NapdU
  misc_feature (31)..(31): n is NapdU
acggnacgannnncagnngganccagnaccn

SEQ ID NO: 32 - DNA, 30 bp, Artificial Sequence
  Synthetic 5406-56_35
  misc_feature (4)..(4): n is NapdU
  misc_feature (9)..(12): n is NapdU
  misc_feature (16)..(17): n is NapdU
  misc_feature (21)..(21): n is NapdU
  misc_feature (26)..(26): n is NapdU
  misc_feature (30)..(30): n is NapdU
cggnacgannnncagnngganccagnaccn

SEQ ID NO: 33 - DNA, 29 bp, Artificial Sequence
  Synthetic 5406-56_36
  misc_feature (3)..(3): n is NapdU
  misc_feature (8)..(11): n is NapdU
  misc_feature (15)..(16): n is NapdU
  misc_feature (20)..(20): n is NapdU
  misc_feature (25)..(25): n is NapdU
  misc_feature (29)..(29): n is NapdU
ggnacgannnncagnngganccagnaccn

SEQ ID NO: 34 - DNA, 28 bp, Artificial Sequence
  Synthetic 5406-56_37
  misc_feature (2)..(2): n is NapdU
  misc_feature (7)..(10): n is NapdU
  misc_feature (14)..(15): n is NapdU
  misc_feature (19)..(19): n is NapdU
  misc_feature (24)..(24): n is NapdU
  misc_feature (28)..(28): n is NapdU
gnacgannnncagnngganccagnaccn

SEQ ID NO: 35 - DNA, 27 bp, Artificial Sequence
  Synthetic 5406-56_38
  misc_feature (1)..(1): n is NapdU
  misc_feature (6)..(9): n is NapdU
  misc_feature (13)..(14): n is NapdU
  misc_feature (18)..(18): n is NapdU
  misc_feature (23)..(23): n is NapdU
  misc_feature (27)..(27): n is NapdU
nacgannnncagnngganccagnaccn

SEQ ID NO: 36 - DNA, 31 bp, Artificial Sequence
  Synthetic 5406-56_39
  misc_feature (1)..(1): n is NapdU
  misc_feature (6)..(6): n is NapdU
  misc_feature (11)..(14): n is NapdU
  misc_feature (18)..(19): n is NapdU
  misc_feature (23)..(23): n is NapdU
  misc_feature (28)..(28): n is NapdU
nacggnacgannnncagnngganccagnacc

SEQ ID NO: 37 - DNA, 30 bp, Artificial Sequence
  Synthetic 5406-56_40
  misc_feature (5)..(5): n is NapdU
  misc_feature (10)..(13): n is NapdU
  misc_feature (17)..(18): n is NapdU
  misc_feature (22)..(22): n is NapdU
  misc_feature (27)..(27): n is NapdU
acggnacgannnncagnngganccagnacc

SEQ ID NO: 38 - DNA, 29 bp, Artificial Sequence
  Synthetic 5406-56_41
  misc_feature (4)..(4): n is NapdU
  misc_feature (9)..(12): n is NapdU
  misc_feature (16)..(17): n is NapdU
  misc_feature (21)..(21): n is NapdU
  misc_feature (26)..(26): n is NapdU
cggnacgannnncagnngganccagnacc

SEQ ID NO: 39 - DNA, 28 bp, Artificial Sequence
  Synthetic 5406-56_42
  misc_feature (3)..(3): n is NapdU
  misc_feature (8)..(11): n is NapdU
  misc_feature (15)..(16): n is NapdU
  misc_feature (20)..(20): n is NapdU
  misc_feature (25)..(25): n is NapdU
ggnacgannnncagnngganccagnacc

SEQ ID NO: 40 - DNA, 27 bp, Artificial Sequence
  Synthetic 5406-56_43
  misc_feature (2)..(2): n is NapdU
  misc_feature (7)..(10): n is NapdU
  misc_feature (14)..(15): n is NapdU
  misc_feature (19)..(19): n is NapdU
  misc_feature (24)..(24): n is NapdU
gnacgannnncagnngganccagnacc

SEQ ID NO: 41 - DNA, 26 bp, Artificial Sequence
  Synthetic 5406-56_44
  misc_feature (1)..(1): n is NapdU
  misc_feature (6)..(9): n is NapdU
  misc_feature (13)..(14): n is NapdU
  misc_feature (18)..(18): n is NapdU
  misc_feature (23)..(23): n is NapdU
nacgannnncagnngganccagnacc

SEQ ID NO: 42 - DNA, 30 bp, Artificial Sequence
  Synthetic 5406-56_45
  misc_feature (1)..(1): n is NapdU
  misc_feature (6)..(6): n is NapdU
  misc_feature (11)..(14): n is NapdU
  misc_feature (18)..(19): n is NapdU
  misc_feature (23)..(23): n is NapdU
  misc_feature (28)..(28): n is NapdU
nacggnacgannnncagnngganccagnac

SEQ ID NO: 43 - DNA, 29 bp, Artificial Sequence
  Synthetic 5406-56_46
  misc_feature (5)..(5): n is NapdU
  misc_feature (10)..(13): n is NapdU
  misc_feature (17)..(18): n is NapdU
  misc_feature (22)..(22): n is NapdU
  misc_feature (27)..(27): n is NapdU
acggnacgannnncagnngganccagnac

SEQ ID NO: 44 - DNA, 28 bp, Artificial Sequence
  Synthetic 5406-56_47
  misc_feature (4)..(4): n is NapdU
  misc_feature (9)..(12): n is NapdU
  misc_feature (16)..(17): n is NapdU
  misc_feature (21)..(21): n is NapdU
  misc_feature (26)..(26): n is NapdU
cggnacgannnncagnngganccagnac

SEQ ID NO: 45 - DNA, 27 bp, Artificial Sequence
  Synthetic 5406-56_48
  misc_feature (3)..(3): n is NapdU
  misc_feature (8)..(11): n is NapdU
  misc_feature (15)..(16): n is NapdU
  misc_feature (20)..(20): n is NapdU
  misc_feature (25)..(25): n is NapdU
ggnacgannnncagnngganccagnac

SEQ ID NO: 46 - DNA, 26 bp, Artificial Sequence
  Synthetic 5406-56_49
  misc_feature (2)..(2): n is NapdU
  misc_feature (7)..(10): n is NapdU
  misc_feature (14)..(15): n is NapdU
  misc_feature (19)..(19): n is NapdU
  misc_feature (24)..(24): n is NapdU
gnacgannnncagnngganccagnac

SEQ ID NO: 47 - DNA, 25 bp, Artificial Sequence
  Synthetic 5406-56_50
  misc_feature (1)..(1): n is NapdU
  misc_feature (6)..(9): n is NapdU
  misc_feature (13)..(14): n is NapdU
  misc_feature (18)..(18): n is NapdU
  misc_feature (23)..(23): n is NapdU
nacgannnncagnngganccagnac

SEQ ID NO: 48 - DNA, 50 bp, Artificial Sequence
  Synthetic 5334-8_3
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
  misc_feature (41)..(42): n is 2NapdU
  misc_feature (44)..(44): n is 2NapdU
cggcacnccacanccaagacngcggnnaccnngnaggcgannangggcag

SEQ ID NO: 49 - DNA, 48 bp, Artificial Sequence
  Synthetic 5334-8_4
  misc_feature (5)..(5): n is 2NapdU
  misc_feature (11)..(11): n is 2NapdU
  misc_feature (19)..(19): n is 2NapdU
  misc_feature (24)..(25): n is 2NapdU
  misc_feature (29)..(30): n is 2NapdU
  misc_feature (32)..(32): n is 2NapdU
  misc_feature (39)..(40): n is 2NapdU
  misc_feature (42)..(42): n is 2NapdU
gcacnccacanccaagacngcggnnaccnngnaggcgannangggcag

SEQ ID NO: 50 - DNA, 46 bp, Artificial Sequence
  Synthetic 5334-8_5
  misc_feature (3)..(3): n is 2NapdU
  misc_feature (9)..(9): n is 2NapdU
  misc_feature (17)..(17): n is 2NapdU
  misc_feature (22)..(23): n is 2NapdU
  misc_feature (27)..(28): n is 2NapdU
  misc_feature (30)..(30): n is 2NapdU
  misc_feature (37)..(38): n is 2NapdU
  misc_feature (40)..(40): n is 2NapdU
acnccacanccaagacngcggnnaccnngnaggcgannangggcag

SEQ ID NO: 51 - DNA, 44 bp, Artificial Sequence
  Synthetic 5334-8_6
  misc_feature (1)..(1): n is 2NapdU
  misc_feature (7)..(7): n is 2NapdU
  misc_feature (15)..(15): n is 2NapdU
  misc_feature (20)..(21): n is 2NapdU
  misc_feature (25)..(26): n is 2NapdU
  misc_feature (28)..(28): n is 2NapdU
  misc_feature (35)..(36): n is 2NapdU
  misc_feature (38)..(38): n is 2NapdU
nccacanccaagacngcggnnaccnngnaggcgannangggcag

SEQ ID NO: 52 - DNA, 42 bp, Artificial Sequence
  Synthetic 5334-8_7
  misc_feature (5)..(5): n is 2NapdU
  misc_feature (13)..(13): n is 2NapdU
  misc_feature (18)..(19): n is 2NapdU
  misc_feature (23)..(24): n is 2NapdU
  misc_feature (26)..(26): n is 2NapdU
  misc_feature (33)..(34): n is 2NapdU
  misc_feature (36)..(36): n is 2NapdU
cacanccaagacngcggnnaccnngnaggcgannangggcag

SEQ ID NO: 53 - DNA, 40 bp, Artificial Sequence
  Synthetic 5334-8_8
  misc_feature (3)..(3): n is 2NapdU
  misc_feature (11)..(11): n is 2NapdU
  misc_feature (16)..(17): n is 2NapdU
  misc_feature (21)..(22): n is 2NapdU
  misc_feature (24)..(24): n is 2NapdU
  misc_feature (31)..(32): n is 2NapdU
  misc_feature (34)..(34): n is 2NapdU
canccaagacngcggnnaccnngnaggcgannangggcag

SEQ ID NO: 54 - DNA, 38 bp, Artificial Sequence
  Synthetic 5334-8_9
  misc_feature (1)..(1): n is 2NapdU
  misc_feature (9)..(9): n is 2NapdU
  misc_feature (14)..(15): n is 2NapdU
  misc_feature (19)..(20): n is 2NapdU
  misc_feature (22)..(22): n is 2NapdU
  misc_feature (29)..(30): n is 2NapdU
  misc_feature (32)..(32): n is 2NapdU
nccaagacngcggnnaccnngnaggcgannangggcag

SEQ ID NO: 55 - DNA, 36 bp, Artificial Sequence
  Synthetic 5334-8_10
  misc_feature (7)..(7): n is 2NapdU
  misc_feature (12)..(13): n is 2NapdU
  misc_feature (17)..(18): n is 2NapdU
  misc_feature (20)..(20): n is 2NapdU
  misc_feature (27)..(28): n is 2NapdU
  misc_feature (30)..(30): n is 2NapdU
caagacngcggnnaccnngnaggcgannangggcag

SEQ ID NO: 56 - DNA, 34 bp, Artificial Sequence
  Synthetic 5334-8_11
  misc_feature (5)..(5): n is 2NapdU
  misc_feature (10)..(11): n is 2NapdU
  misc_feature (15)..(16): n is 2NapdU
  misc_feature (18)..(18): n is 2NapdU
  misc_feature (25)..(26): n is 2NapdU
  misc_feature (28)..(28): n is 2NapdU
agacngcggnnaccnngnaggcgannangggcag

SEQ ID NO: 57 - DNA, 48 bp, Artificial Sequence
  Synthetic 5334-8_12
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
  misc_feature (41)..(42): n is 2NapdU
  misc_feature (44)..(44): n is 2NapdU
cggcacnccacanccaagacngcggnnaccnngnaggcgannangggc

SEQ ID NO: 58 - DNA, 46 bp, Artificial Sequence
  Synthetic 5334-8_13
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
  misc_feature (41)..(42): n is 2NapdU
  misc_feature (44)..(44): n is 2NapdU
cggcacnccacanccaagacngcggnnaccnngnaggcgannangg

SEQ ID NO: 59 - DNA, 44 bp, Artificial Sequence
  Synthetic 5334-8_14
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
  misc_feature (41)..(42): n is 2NapdU
  misc_feature (44)..(44): n is 2NapdU
cggcacnccacanccaagacngcggnnaccnngnaggcgannan

SEQ ID NO: 60 - DNA, 42 bp, Artificial Sequence
  Synthetic 5334-8_15
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
  misc_feature (41)..(42): n is 2NapdU
cggcacnccacanccaagacngcggnnaccnngnaggcgann

SEQ ID NO: 61 - DNA, 40 bp, Artificial Sequence
  Synthetic 5334-8_16
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
cggcacnccacanccaagacngcggnnaccnngnaggcga

SEQ ID NO: 62 - DNA, 38 bp, Artificial Sequence
  Synthetic 5334-8_17
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
cggcacnccacanccaagacngcggnnaccnngnaggc

SEQ ID NO: 63 - DNA, 36 bp, Artificial Sequence
  Synthetic 5334-8_18
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
cggcacnccacanccaagacngcggnnaccnngnag

SEQ ID NO: 64 - DNA, 34 bp, Artificial Sequence
  Synthetic 5334-8_19
  misc_feature (7)..(7): n is 2NapdU
  misc_feature (13)..(13): n is 2NapdU
  misc_feature (21)..(21): n is 2NapdU
  misc_feature (26)..(27): n is 2NapdU
  misc_feature (31)..(32): n is 2NapdU
  misc_feature (34)..(34): n is 2NapdU
cggcacnccacanccaagacngcggnnaccnngn

SEQ ID NO: 65 - DNA, 33 bp, Artificial Sequence
  Synthetic 5334-8_21
  misc_feature (4)..(4): n is 2NapdU
  misc_feature (9)..(10): n is 2NapdU
  misc_feature (14)..(15): n is 2NapdU
  misc_feature (17)..(17): n is 2NapdU
  misc_feature (24)..(25): n is 2NapdU
  misc_feature (27)..(27): n is 2NapdU
gacngcggnnaccnngnaggcgannangggcag

SEQ ID NO: 66 - DNA, 32 bp, Artificial Sequence
  Synthetic 5334-8_22
  misc_feature (3)..(3): n is 2NapdU
  misc_feature (8)..(9): n is 2NapdU
  misc_feature (13)..(14): n is 2NapdU
  misc_feature (16)..(16): n is 2NapdU
  misc_feature (23)..(24): n is 2NapdU
  misc_feature (26)..(26): n is 2NapdU
acngcggnnaccnngnaggcgannangggcag

SEQ ID NO: 67 - DNA, 31 bp, Artificial Sequence
  Synthetic 5334-8_23
  misc_feature (2)..(2): n is 2NapdU
  misc_feature (7)..(8): n is 2NapdU
  misc_feature (12)..(13): n is 2NapdU
  misc_feature (15)..(15): n is 2NapdU
  misc_feature (22)..(23): n is 2NapdU
  misc_feature (25)..(25): n is 2NapdU
cngcggnnaccnngnaggcgannangggcag

SEQ ID NO: 68 - DNA, 30 bp, Artificial Sequence
  Synthetic 5334-8_24
  misc_feature (1)..(1): n is 2NapdU
  misc_feature (6)..(7): n is 2NapdU
  misc_feature (11)..(12): n is 2NapdU
  misc_feature (14)..(14): n is 2NapdU
  misc_feature (21)..(22): n is 2NapdU
  misc_feature (24)..(24): n is 2NapdU
ngcggnnaccnngnaggcgannangggcag

SEQ ID NO: 69 - DNA, 29 bp, Artificial Sequence
  Synthetic 5334-8_25
  misc_feature (5)..(6): n is 2NapdU
  misc_feature (10)..(11): n is 2NapdU
  misc_feature (13)..(13): n is 2NapdU
  misc_feature (20)..(21): n is 2NapdU
  misc_feature (23)..(23): n is 2NapdU
gcggnnaccnngnaggcgannangggcag

SEQ ID NO: 70 - DNA, 28 bp, Artificial Sequence
  Synthetic 5334-8_26
  misc_feature (4)..(5): n is 2NapdU
  misc_feature (9)..(10): n is 2NapdU
  misc_feature (12)..(12): n is 2NapdU
  misc_feature (19)..(20): n is 2NapdU
  misc_feature (22)..(22): n is 2NapdU
cggnnaccnngnaggcgannangggcag

SEQ ID NO: 71 - DNA, 27 bp, Artificial Sequence
  Synthetic 5334-8_27
  misc_feature (3)..(4): n is 2NapdU
  misc_feature (8)..(9): n is 2NapdU
  misc_feature (11)..(11): n is 2NapdU
  misc_feature (18)..(19): n is 2NapdU
  misc_feature (21)..(21): n is 2NapdU
ggnnaccnngnaggcgannangggcag

SEQ ID NO: 72 - DNA, 33 bp, Artificial Sequence
  Synthetic 5334-8_28
  misc_feature (5)..(5): n is 2NapdU
  misc_feature (10)..(11): n is 2NapdU
  misc_feature (15)..(16): n is 2NapdU
  misc_feature (18)..(18): n is 2NapdU
  misc_feature (25)..(26): n is 2NapdU
  misc_feature (28)..(28): n is 2NapdU
agacngcggnnaccnngnaggcgannangggca

SEQ ID NO: 73 - DNA, 32 bp, Artificial Sequence
  Synthetic 5334-8_29
  misc_feature (5)..(5): n is 2NapdU
  misc_feature (10)..(11): n is 2NapdU
  misc_feature (15)..(16): n is 2NapdU
  misc_feature (18)..(18): n is 2NapdU
  misc_feature (25)..(26): n is 2NapdU
  misc_feature (28)..(28): n is 2NapdU
agacngcggnnaccnngnaggcgannangggc

SEQ ID NO: 74 - DNA, 31 bp, Artificial Sequence
  Synthetic 5334-8_30
  misc_feature (5)..(5): n is 2NapdU
  misc_feature (10)..(11): n is 2NapdU
  misc_feature (15)..(16): n is 2NapdU
  misc_feature (18)..(18): n is 2NapdU
  misc_feature (25)..(26): n is 2NapdU
  misc_feature (28)..(28): n is 2NapdU
agacngcggnnaccnngnaggcgannanggg

SEQ ID NO: 75 - DNA, 30 bp, Artificial Sequence
  Synthetic 5334-8_31
  misc_feature (5)..(5): n is 2NapdU
  misc_feature (10)..(11): n is 2NapdU
  misc_feature (15)..(16): n is 2NapdU
  misc_feature (18)..(18): n is 2NapdU
  misc_feature (25)..(26): n is 2NapdU
  misc_feature (28)..(28): n is 2NapdU
agacngcggnnaccnngnaggcgannangg

SEQ ID NO: 76 - DNA, 28 bp, Artificial Sequence
  Synthetic 5334-8_32
  misc_feature (5)..(5): n is 2NapdU
  misc_feature (10)..(11): n is 2NapdU
  misc_feature (15)..(16): n is 2NapdU
  misc_feature (18)..(18): n is 2NapdU
  misc_feature (25)..(26): n is 2NapdU
  misc_feature (28)..(28): n is 2NapdU
agacngcggnnaccnngnaggcgannan

SEQ ID NO: 77 - DNA, 26 bp, Artificial Sequence
  Synthetic 5334-8_33
  misc_feature (5)..(5): n is 2NapdU
  misc_feature (10)..(11): n is 2NapdU
  misc_feature (15)..(16): n is 2NapdU
  misc_feature (18)..(18): n is 2NapdU
  misc_feature (25)..(26): n is 2NapdU
agacngcggnnaccnngnaggcgann

SEQ ID NO: 78 - DNA, 28 bp, Artificial Sequence
  Synthetic 5334-8_34
  misc_feature (5)..(6): n is 2NapdU
  misc_feature (10)..(11): n is 2NapdU
  misc_feature (13)..(13): n is 2NapdU
  misc_feature (20)..(21): n is 2NapdU
  misc_feature (23)..(23): n is 2NapdU
gcggnnaccnngnaggcgannangggca

SEQ ID NO: 79 - DNA, 50 bp, Artificial Sequence
  Synthetic 14125-144_3
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
  misc_feature (35)..(35): n is NapdU
  misc_feature (37)..(38): n is NapdU
  misc_feature (42)..(42): n is NapdU
gcctgcacganggcgaacncccngaangcncnngncnnaagnccccaaca

SEQ ID NO: 80 - DNA, 48 bp, Artificial Sequence
  Synthetic 14125-144_4
  misc_feature (9)..(9): n is NapdU
  misc_feature (17)..(17): n is NapdU
  misc_feature (21)..(21): n is NapdU
  misc_feature (25)..(25): n is NapdU
  misc_feature (28)..(28): n is NapdU
  misc_feature (30)..(31): n is NapdU
  misc_feature (33)..(33): n is NapdU
  misc_feature (35)..(36): n is NapdU
  misc_feature (40)..(40): n is NapdU
ctgcacganggcgaacncccngaangcncnngncnnaagnccccaaca

SEQ ID NO: 81 - DNA, 46 bp, Artificial Sequence
  Synthetic 14125-144_5
  misc_feature (7)..(7): n is NapdU
  misc_feature (15)..(15): n is NapdU
  misc_feature (19)..(19): n is NapdU
  misc_feature (23)..(23): n is NapdU
  misc_feature (26)..(26): n is NapdU
  misc_feature (28)..(29): n is NapdU
  misc_feature (31)..(31): n is NapdU
  misc_feature (33)..(34): n is NapdU
  misc_feature (38)..(38): n is NapdU
gcacganggcgaacncccngaangcncnngncnnaagnccccaaca

SEQ ID NO: 82 - DNA, 44 bp, Artificial Sequence
  Synthetic 14125-144_6
  misc_feature (5)..(5): n is NapdU
  misc_feature (13)..(13): n is NapdU
  misc_feature (17)..(17): n is NapdU
  misc_feature (21)..(21): n is NapdU
  misc_feature (24)..(24): n is NapdU
  misc_feature (26)..(27): n is NapdU
  misc_feature (29)..(29): n is NapdU
  misc_feature (31)..(32): n is NapdU
  misc_feature (36)..(36): n is NapdU
acganggcgaacncccngaangcncnngncnnaagnccccaaca

SEQ ID NO: 83 - DNA, 42 bp, Artificial Sequence
  Synthetic 14125-144_7
  misc_feature (3)..(3): n is NapdU
  misc_feature (11)..(11): n is NapdU
  misc_feature (15)..(15): n is NapdU
  misc_feature (19)..(19): n is NapdU
  misc_feature (22)..(22): n is NapdU
  misc_feature (24)..(25): n is NapdU
  misc_feature (27)..(27): n is NapdU
  misc_feature (29)..(30): n is NapdU
  misc_feature (34)..(34): n is NapdU
ganggcgaacncccngaangcncnngncnnaagnccccaaca

SEQ ID NO: 84 - DNA, 40 bp, Artificial Sequence
  Synthetic 14125-144_8
  misc_feature (1)..(1): n is NapdU
  misc_feature (9)..(9): n is NapdU
  misc_feature (13)..(13): n is NapdU
  misc_feature (17)..(17): n is NapdU
  misc_feature (20)..(20): n is NapdU
  misc_feature (22)..(23): n is NapdU
  misc_feature (25)..(25): n is NapdU
  misc_feature (27)..(28): n is NapdU
  misc_feature (32)..(32): n is NapdU
nggcgaacncccngaangcncnngncnnaagnccccaaca

SEQ ID NO: 85 - DNA, 38 bp, Artificial Sequence
  Synthetic 14125-144_9
  misc_feature (7)..(7): n is NapdU
  misc_feature (11)..(11): n is NapdU
  misc_feature (15)..(15): n is NapdU
  misc_feature (18)..(18): n is NapdU
  misc_feature (20)..(21): n is NapdU
  misc_feature (23)..(23): n is NapdU
  misc_feature (25)..(26): n is NapdU
  misc_feature (30)..(30): n is NapdU
gcgaacncccngaangcncnngncnnaagnccccaaca

SEQ ID NO: 86 - DNA, 36 bp, Artificial Sequence
  Synthetic 14125-144_10
  misc_feature (5)..(5): n is NapdU
  misc_feature (9)..(9): n is NapdU
  misc_feature (13)..(13): n is NapdU
  misc_feature (16)..(16): n is NapdU
  misc_feature (18)..(19): n is NapdU
  misc_feature (21)..(21): n is NapdU
  misc_feature (23)..(24): n is NapdU
  misc_feature (28)..(28): n is NapdU
gaacncccngaangcncnngncnnaagnccccaaca

SEQ ID NO: 87 - DNA, 34 bp, Artificial Sequence
  Synthetic 14125-144_11
  misc_feature (3)..(3): n is NapdU
  misc_feature (7)..(7): n is NapdU
  misc_feature (11)..(11): n is NapdU
  misc_feature (14)..(14): n is NapdU
  misc_feature (16)..(17): n is NapdU
  misc_feature (19)..(19): n is NapdU
  misc_feature (21)..(22): n is NapdU
  misc_feature (26)..(26): n is NapdU
acncccngaangcncnngncnnaagnccccaaca

SEQ ID NO: 88 - DNA, 48 bp, Artificial Sequence
  Synthetic 14125-144_12
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
  misc_feature (35)..(35): n is NapdU
  misc_feature (37)..(38): n is NapdU
  misc_feature (42)..(42): n is NapdU
gcctgcacganggcgaacncccngaangcncnngncnnaagnccccaa

SEQ ID NO: 89 - DNA, 46 bp, Artificial Sequence
  Synthetic 14125-144_13
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
  misc_feature (35)..(35): n is NapdU
  misc_feature (37)..(38): n is NapdU
  misc_feature (42)..(42): n is NapdU
gcctgcacganggcgaacncccngaangcncnngncnnaagncccc

SEQ ID NO: 90 - DNA, 44 bp, Artificial Sequence
  Synthetic 14125-144_14
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
  misc_feature (35)..(35): n is NapdU
  misc_feature (37)..(38): n is NapdU
  misc_feature (42)..(42): n is NapdU
gcctgcacganggcgaacncccngaangcncnngncnnaagncc

SEQ ID NO: 91 - DNA, 42 bp, Artificial Sequence
  Synthetic 14125-144_15
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
  misc_feature (35)..(35): n is NapdU
  misc_feature (37)..(38): n is NapdU
  misc_feature (42)..(42): n is NapdU
gcctgcacganggcgaacncccngaangcncnngncnnaagn

SEQ ID NO: 92 - DNA, 40 bp, Artificial Sequence
  Synthetic 14125-144_16
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
  misc_feature (35)..(35): n is NapdU
  misc_feature (37)..(38): n is NapdU
gcctgcacganggcgaacncccngaangcncnngncnnaa

SEQ ID NO: 93 - DNA, 38 bp, Artificial Sequence
  Synthetic 14125-144_17
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
  misc_feature (35)..(35): n is NapdU
  misc_feature (37)..(38): n is NapdU
gcctgcacganggcgaacncccngaangcncnngncnn

SEQ ID NO: 94 - DNA, 36 bp, Artificial Sequence
  Synthetic 14125-144_18
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
  misc_feature (35)..(35): n is NapdU
gcctgcacganggcgaacncccngaangcncnngnc

SEQ ID NO: 95 - DNA, 34 bp, Artificial Sequence
  Synthetic 14125-144_19
  misc_feature (11)..(11): n is NapdU
  misc_feature (19)..(19): n is NapdU
  misc_feature (23)..(23): n is NapdU
  misc_feature (27)..(27): n is NapdU
  misc_feature (30)..(30): n is NapdU
  misc_feature (32)..(33): n is NapdU
gcctgcacganggcgaacncccngaangcncnng

SEQ ID NO: 96 - DNA, 32 bp, Artificial Sequence
  Synthetic 14125-144_20
  misc_feature (3)..(3): n is NapdU
  misc_feature (11)..(11): n is NapdU
  misc_feature (15)..(15): n is NapdU
  misc_feature (19)..(19): n is NapdU
  misc_feature (22)..(22): n is NapdU
  misc_feature (24)..(25): n is NapdU
  misc_feature (27)..(27): n is NapdU
  misc_feature (29)..(30): n is NapdU
ganggcgaacncccngaangcncnngncnnaa

SEQ ID NO: 97 - DNA, 31 bp, Artificial Sequence
  Synthetic 14125-144_21
  misc_feature (2)..(2): n is NapdU
  misc_feature (10)..(10): n is NapdU
  misc_feature (14)..(14): n is NapdU
  misc_feature (18)..(18): n is NapdU
  misc_feature (21)..(21): n is NapdU
  misc_feature (23)..(24): n is NapdU
  misc_feature (26)..(26): n is NapdU
  misc_feature (28)..(29): n is NapdU
anggcgaacncccngaangcncnngncnnaa

SEQ ID NO: 98 - DNA, 30 bp, Artificial Sequence
  Synthetic 14125-144_22
  misc_feature (1)..(1): n is NapdU
  misc_feature (9)..(9): n is NapdU
  misc_feature (13)..(13): n is NapdU
  misc_feature (17)..(17): n is NapdU
  misc_feature (20)..(20): n is NapdU
  misc_feature (22)..(23): n is NapdU
  misc_feature (25)..(25): n is NapdU
  misc_feature (27)..(28): n is NapdU
nggcgaacncccngaangcncnngncnnaa

SEQ ID NO: 99 - DNA, 29 bp, Artificial Sequence
  Synthetic 14125-144_23
  misc_feature (8)..(8): n is NapdU
  misc_feature (12)..(12): n is NapdU
  misc_feature (16)..(16): n is NapdU
  misc_feature (19)..(19): n is NapdU
  misc_feature (21)..(22): n is NapdU
  misc_feature (24)..(24): n is NapdU
  misc_feature (26)..(27): n is NapdU
ggcgaacncccngaangcncnngncnnaa

SEQ ID NO: 100 - DNA, 31 bp, Artificial Sequence
  Synthetic 14125-144_24
  misc_feature (3)..(3): n is NapdU
  misc_feature (11)..(11): n is NapdU
  misc_feature (15)..(15): n is NapdU
  misc_feature (19)..(19): n is NapdU
  misc_feature (22)..(22): n is NapdU
  misc_feature (24)..(25): n is NapdU
  misc_feature (27)..(27): n is NapdU
  misc_feature (29)..(30): n is NapdU
ganggcgaacncccngaangcncnngncnna

SEQ ID NO: 101 - DNA, 30 bp, Artificial Sequence
  Synthetic 14125-144_25
  misc_feature (2)..(2): n is NapdU
  misc_feature (10)..(10): n is NapdU
  misc_feature (14)..(14): n is NapdU
  misc_feature (18)..(18): n is NapdU
  misc_feature (21)..(21): n is NapdU
  misc_feature (23)..(24): n is NapdU
  misc_feature (26)..(26): n is NapdU
  misc_feature (28)..(29): n is NapdU
anggcgaacncccngaangcncnngncnna

SEQ ID NO: 102 - DNA, 29 bp, Artificial Sequence
  Synthetic 14125-144_26
  misc_feature (1)..(1): n is NapdU
  misc_feature (9)..(9): n is NapdU
  misc_feature (13)..(13): n is NapdU
  misc_feature (17)..(17): n is NapdU
  misc_feature (20)..(20): n is NapdU
  misc_feature (22)..(23): n is NapdU
  misc_feature (25)..(25): n is NapdU
  misc_feature (27)..(28): n is NapdU
nggcgaacncccngaangcncnngncnna

SEQ ID NO: 103 - DNA, 28 bp, Artificial Sequence
  Synthetic 14125-144_27
  misc_feature (8)..(8): n is NapdU
  misc_feature (12)..(12): n is NapdU
  misc_feature (16)..(16): n is NapdU
  misc_feature (19)..(19): n is NapdU
  misc_feature (21)..(22): n is NapdU
  misc_feature (24)..(24): n is NapdU
  misc_feature (26)..(27): n is NapdU
ggcgaacncccngaangcncnngncnna

SEQ ID NO: 104 - DNA, 30 bp, Artificial Sequence
  Synthetic 14125-144_28
  misc_feature (3)..(3): n is NapdU
  misc_feature (11)..(11): n is NapdU
  misc_feature (15)..(15): n is NapdU
  misc_feature (19)..(19): n is NapdU
  misc_feature (22)..(22): n is NapdU
  misc_feature (24)..(25): n is NapdU
  misc_feature (27)..(27): n is NapdU
  misc_feature (29)..(30): n is NapdU
ganggcgaacncccngaangcncnngncnn

SEQ ID NO: 105 - DNA, 29 bp, Artificial Sequence
  Synthetic 14125-144_29
  misc_feature (2)..(2): n is NapdU
  misc_feature (10)..(10): n is NapdU
  misc_feature (14)..(14): n is NapdU
  misc_feature (18)..(18): n is NapdU
  misc_feature (21)..(21): n is NapdU
  misc_feature (23)..(24): n is NapdU
  misc_feature (26)..(26): n is NapdU
  misc_feature (28)..(29): n is NapdU
anggcgaacncccngaangcncnngncnn

SEQ ID NO: 106 - DNA, 28 bp, Artificial Sequence
  Synthetic 14125-144_30
  misc_feature (1)..(1): n is NapdU
  misc_feature (9)..(9): n is NapdU
  misc_feature (13)..(13): n is NapdU
  misc_feature (17)..(17): n is NapdU
  misc_feature (20)..(20): n is NapdU
  misc_feature (22)..(23): n is NapdU
  misc_feature (25)..(25): n is NapdU
  misc_feature (27)..(28): n is NapdU
nggcgaacncccngaangcncnngncnn

SEQ ID NO: 107 - DNA, 27 bp, Artificial Sequence
  Synthetic 14125-144_31
  misc_feature (8)..(8): n is NapdU
  misc_feature (12)..(12): n is NapdU
  misc_feature (16)..(16): n is NapdU
  misc_feature (19)..(19): n is NapdU
  misc_feature (21)..(22): n is NapdU
  misc_feature (24)..(24): n is NapdU
  misc_feature (26)..(27): n is NapdU
ggcgaacncccngaangcncnngncnn

SEQ ID NO: 108 - DNA, 29 bp, Artificial Sequence
  Synthetic 14125-144_32
  misc_feature (3)..(3): n is NapdU
  misc_feature (11)..(11): n is NapdU
  misc_feature (15)..(15): n is NapdU
  misc_feature (19)..(19): n is NapdU
  misc_feature (22)..(22): n is NapdU
  misc_feature (24)..(25): n is NapdU
  misc_feature (27)..(27): n is NapdU
  misc_feature (29)..(29): n is NapdU
ganggcgaacncccngaangcncnngncn

SEQ ID NO: 109 - DNA, 28 bp, Artificial Sequence
  Synthetic 14125-144_33
  misc_feature (2)..(2): n is NapdU
  misc_feature (10)..(10): n is NapdU
  misc_feature (14)..(14): n is NapdU
  misc_feature (18)..(18): n is NapdU
  misc_feature (21)..(21): n is NapdU
  misc_feature (23)..(24): n is NapdU
  misc_feature (26)..(26): n is NapdU
  misc_feature (28)..(28): n is NapdU
anggcgaacncccngaangcncnngncn

SEQ ID NO: 110 - DNA, 27 bp, Artificial Sequence
  Synthetic 14125-144_34
  misc_feature (1)..(1): n is NapdU
  misc_feature (9)..(9): n is NapdU
  misc_feature (13)..(13): n is NapdU
  misc_feature (17)..(17): n is NapdU
  misc_feature (20)..(20): n is NapdU
  misc_feature (22)..(23): n is NapdU
  misc_feature (25)..(25): n is NapdU
  misc_feature (27)..(27): n is NapdU
nggcgaacncccngaangcncnngncn

SEQ ID NO: 111 - DNA, 26 bp, Artificial Sequence
  Synthetic 14125-144_35
  misc_feature (8)..(8): n is NapdU
  misc_feature (12)..(12): n is NapdU
  misc_feature (16)..(16): n is NapdU
  misc_feature (19)..(19): n is NapdU
  misc_feature (21)..(22): n is NapdU
  misc_feature (24)..(24): n is NapdU
  misc_feature (26)..(26): n is NapdU
ggcgaacncccngaangcncnngncn

SEQ ID NO: 112 - DNA, 28 bp, Artificial Sequence
  Synthetic 14125-144_36
  misc_feature (3)..(3): n is NapdU
  misc_feature (11)..(11): n is NapdU
  misc_feature (15)..(15): n is NapdU
  misc_feature (19)..(19): n is NapdU
  misc_feature (22)..(22): n is NapdU
  misc_feature (24)..(25): n is NapdU
  misc_feature (27)..(27): n is NapdU
ganggcgaacncccngaangcncnngnc

SEQ ID NO: 113 - DNA, 27 bp, Artificial Sequence
  Synthetic 14125-144_37
  misc_feature (2)..(2): n is NapdU
  misc_feature (10)..(10): n is NapdU
  misc_feature (14)..(14): n is NapdU
  misc_feature (18)..(18): n is NapdU
  misc_feature (21)..(21): n is NapdU
  misc_feature (23)..(24): n is NapdU
  misc_feature (26)..(26): n is NapdU
anggcgaacncccngaangcncnngnc

SEQ ID NO: 114 - DNA, 26 bp, Artificial Sequence
  Synthetic 14125-144_38
  misc_feature (1)..(1): n is NapdU
  misc_feature (9)..(9): n is NapdU
  misc_feature (13)..(13): n is NapdU
  misc_feature (17)..(17): n is NapdU
  misc_feature (20)..(20): n is NapdU
  misc_feature (22)..(23): n is NapdU
  misc_feature (25)..(25): n is NapdU
nggcgaacncccngaangcncnngnc

SEQ ID NO: 115 - DNA, 25 bp, Artificial Sequence
  Synthetic 14125-144_39
  misc_feature (8)..(8): n is NapdU
  misc_feature (12)..(12): n is NapdU
  misc_feature (16)..(16): n is NapdU
  misc_feature (19)..(19): n is NapdU
  misc_feature (21)..(22): n is NapdU
  misc_feature (24)..(24): n is NapdU
ggcgaacncccngaangcncnngnc